(19) United States
(12) Patent Application Publication
(10) Pub. No.: US 2004/0043389 A1
(43) Pub. Date: Mar. 4, 2004

(54) Methods and compositions for identifying risk factors for abnormal lipid levels and the diseases and disorders associated therewith

(75) Inventor: Jeanette McCarthy, San Diego, CA (US)

Correspondence Address: MILLENNIUM PHARMACEUTICALS, INC., 40 Landsdowne Street, CAMBRIDGE, MA, 02139, US

(73) Assignee: Vitivity, Inc., Cambridge, MA

(21) Appl. No.: 10/235,192

(22) Filed: Sep. 4, 2002

(51) Int. Cl. C12Q 1/68

(52) U.S. Cl. 435/6

(57) ABSTRACT

The present invention is based at least in part on the discovery of associations between polymorphic regions and specific diseases or disorders, e.g., abnormal lipid levels, e.g., abnormally low HDL-C levels, or diseases or disorders associated with abnormal lipid levels, e.g., vascular or metabolic diseases or disorders. Accordingly, the invention provides nucleic acid molecules having a nucleotide sequence of an allelic variant of a gene listed in Tables 1-5. The invention also provides methods for identifying specific alleles of polymorphic regions of a gene listed in Tables 1-5, methods for determining whether a subject has or is at risk of developing a disease which is associated with a specific allele of a polymorphic region of a gene listed in Tables 1-5, e.g., abnormal lipid levels, e.g., abnormally low HDL-C levels, or a vascular or metabolic disease or disorder, based on detection of one or more polymorphisms within the genes listed in Tables 1-5, and kits for performing such methods. The invention further provides methods for identifying a subject who has, or is at risk for developing, abnormal lipid levels, e.g., abnormally low HDL-C levels, or a vascular or metabolic disease or disorder, as a candidate for a particular clinical course of therapy or a particular diagnostic evaluation.

### BACKGROUND OF THE INVENTION

Coronary heart disease is a major health risk throughout the industrialized world. Coronary Artery Disease (CAD), or atherosclerosis, the most prevalent of cardiovascular diseases, is the principal cause of heart attack, stroke, and gangrene of the extremities, and thereby the principle cause of death in the United States. CAD involves the progressional narrowing of the arteries due to a build-up of atherosclerotic plaque. Myocardial infarction (MI), e.g., heart attack, results when the heart is damaged due to reduced blood flow to the heart caused by the build-up of plaque in the coronary arteries.

CAD is a complex disease involving many cell types and molecular factors (described in, for example, Ross, 1993, Nature 362: 801-809). The process, in normal circumstances a protective response to insults to the endothelium and smooth muscle cells (SMCs) of the wall of the artery, consists of the formation of fibrofatty and fibrous lesions or plaques, preceded and accompanied by inflammation. The advanced lesions of atherosclerosis may occlude the artery concerned, and result from an excessive inflammatory-fibroproliferative response to numerous different forms of insult. Injury or dysfunction of the vascular endothelium is a common feature of many conditions that predispose a subject to accelerated development of atherosclerotic cardiovascular disease. For example, shear stresses are thought to be responsible for the frequent occurrence of atherosclerotic plaques in regions of the circulatory system where turbulent blood flow occurs, such as branch points and irregular structures.

The first observable event in the formation of an atherosclerotic plaque occurs when blood-borne monocytes adhere to the vascular endothelial layer and transmigrate through to the sub-endothelial space. Adjacent endothelial cells at the same time produce oxidized low density lipoprotein (LDL). These oxidized LDLs are then taken up in large amounts by the monocytes through scavenger receptors expressed on their surfaces. In contrast to the regulated pathway by which native LDL (nLDL) is taken up by nLDL specific receptors, the scavenger pathway of uptake is not regulated by the monocytes.

These lipid-filled monocytes are called foam cells, and are the major constituent of the fatty streak. Interactions between foam cells and the endothelial and SMCs which surround them lead to a state of chronic local inflammation which can eventually lead to smooth muscle cell proliferation and migration, and the formation of a fibrous plaque.

Such plaques occlude the blood vessel concerned and, thus, restrict the flow of blood, resulting in ischemia. Ischemia is a condition characterized by a lack of oxygen supply in tissues of organs due to inadequate perfusion. Such inadequate perfusion can have a number of natural causes, including atherosclerotic or restenotic lesions, anemia, or stroke. Many medical interventions, such as the interruption of the flow of blood during bypass surgery, for example, also lead to ischemia. In addition to sometimes being caused by diseased cardiovascular tissue, ischemia may sometimes affect cardiovascular tissue, such as in ischemic heart disease.

Dyslipidemia is associated with the development of CAD and atherosclerosis. Although historically much emphasis has been placed on total plasma cholesterol levels as a risk factor for coronary heart disease, it has been clearly established that low levels of high density lipoprotein cholesterol (HDL) is an independent risk factor for this disease. Family and twin studies have shown that there are genetic components that affect HDL levels. However, mutations in the main protein components of HDL (ApoA1 and ApoAII) and in the enzymes that are known to be involved in HDL metabolism (e.g., CETP, HL, LPL and LCAT) do not explain all of the genetic factors affecting HDL levels in the general population (J. L. Breslow, in The Metabolic and Molecular Bases of Inherited Disease, C. R. Scriver, A. L. Beaudet, W. Sly, D. Valle, Eds. (McGraw-Hill, New York, 1995), pp 2031-2052; and S. M. Grundy, (1995) J. Am. Med. Assoc. 256: 2849). This finding in combination with the fact that the mechanisms of HDL metabolism are poorly understood, suggests that there are other as yet unknown factors that contribute to the genetic variability of lipid levels, including HDL levels.

It would thus be beneficial to identify polymorphic regions within genes which are associated with abnormal lipid levels, e.g., low HDL levels. It would further be desirable to provide prognostic, diagnostic, pharmacogenomic, and therapeutic methods utilizing the identified polymorphic regions.

### SUMMARY OF THE INVENTION

The present invention is based, at least in part, on the identification of polymorphic regions within several genes which are associated with abnormal lipid levels, e.g., low HDL-C levels (see Tables 1-5, below). Decreased HDL-C level is a well-known risk factor for the development of vascular diseases and disorders, e.g., CAD and MI, and metabolic diseases or disorders, e.g., diabetes or obesity. Accordingly, SNPs in the genes listed in Table 1 can be utilized to predict, in a subject, an increased risk for developing abnormal lipid levels, e.g., low HDL-C levels, or a disease or disorder associated with abnormal lipid levels, e.g., a vascular disease or disorder, e.g., CAD or MI, or a metabolic disease or disorder, e.g., diabetes or obesity. In particular, a subject having a specific allele listed in Table 5 is at an increased risk of having or developing abnormal lipid levels, e.g., low HDL-C levels, or a disease or disorder associated with abnormal lipid levels, e.g., a vascular or metabolic disease or disorder.

Furthermore, the associations between the allelic variants of the genes listed in Tables 1-5 and low HDL-C level are influenced by gender, indicating an interaction with hormonal status. The association of lipids with allelic variants in genes listed in Tables 1-5, is modulated by hormonal status. Therefore, these polymorphisms may be useful in predicting the effect of hormone replacement therapy (HRT) on lipid levels in female subjects, e.g., postmenopausal female subjects, and therefore the risk for developing a vascular or metabolic disease or disorder.

Thus, the invention relates to polymorphic regions and in particular, SNPs identified as described herein, in combination with each other or in combination with other polymorphisms in the genes listed in Tables 1-5, or in other genes. The invention also relates to the use of these SNPs, and other SNPs in the genes listed in Tables 1-5, or in other genes, particularly those in linkage disequilibrium with these SNPs, for diagnosis of abnormal lipid levels, e.g., abnormally low HDL-C level, or a disease or disorder associated with abnormal lipid levels, e.g., a vascular or metabolic disease or disorder. The SNPs identified herein may further be used in the development of new treatments for abnormal lipid levels, e.g., abnormally low HDL-C level, or a disease or disorder associated with abnormal lipid levels, e.g., a vascular or metabolic disease or disorder, based upon comparison of the variant and normal versions of the gene or gene product (e.g., the reference sequence), and development of cell-culture based and animal models for research and treatment of vascular diseases or disorders or metabolic diseases or disorders. The invention further relates to novel compounds and pharmaceutical compositions for use in the diagnosis and treatment of such disorders. In preferred embodiments, the vascular disease is CAD or MI, and the metabolic disease is obesity or diabetes.

The polymorphisms of the invention may thus be used, in combination with each other or with polymorphisms in the genes listed in Tables 1-5, or in other genes, in prognostic, diagnostic, and therapeutic methods. For example, the polymorphisms of the invention can be used to determine whether a subject has, or is, or is not at risk of developing a disease or disorder associated with a specific allelic variant of a polymorphic region of a gene listed in Tables 1-5, e.g., a disease or disorder associated with aberrant gene expression or protein activity, e.g., abnormal lipid levels, e.g., abnormally low HDL-C level, or a disease or disorder associated with abnormal lipid levels, e.g., a vascular or metabolic disease or disorder.

The invention further provides methods for determining at least a portion of a gene listed in Tables 1-5. In a preferred embodiment, the method comprises contacting a sample nucleic acid comprising the sequence of a gene listed in Tables 1-5 with a probe or primer having a sequence which is complementary to the sequence of a gene listed in Tables 1-5, carrying out a reaction that would amplify and/or detect differences in a region of interest within the sequence of a gene listed in Tables 1-5, and comparing the results of each reaction with that of a reaction with a control (known) gene (e.g., a gene listed in Tables 1-5 from a human not afflicted with abnormal lipid levels, e.g., low HDL-C levels, vascular disease or disorder e.g., CAD, MI, or a metabolic disease or disorder, e.g., obesity or diabetes) so as to determine the molecular structure of the gene sequence of a gene listed in Tables 1-5 in the sample nucleic acid. The method of the invention can be used for example in determining the molecular structure of at least a portion of an exon, an intron, a 5′ upstream regulatory element, or the 3′ untranslated region.

In another preferred embodiment, the method comprises determining the nucleotide content of at least a portion of a gene listed in Tables 1-5, such as by sequence analysis. In yet another embodiment, determining the molecular structure of at least a portion of a gene listed in Tables 1-5 is carried out by single-stranded conformation polymorphism (SSCP). In yet another embodiment, the method is an oligonucleotide ligation assay (OLA). Other methods within the scope of the invention for determining the molecular structure of at least a portion of a gene listed in Tables 1-5 include hybridization of allele-specific oligonucleotides, sequence specific amplification, primer specific extension, and denaturing high performance liquid chromatography (DHPLC). In at least some of the methods of the invention, the probe or primer is allele specific. Preferred probes or primers are single stranded nucleic acids, which optionally are labeled.

The methods of the invention can be used for determining the identity of a nucleotide or amino acid residue within a polymorphic region of a human gene listed in Tables 1-5 present in a subject. For example, the methods of the invention can be useful for determining whether a subject has, or is or is not at risk of developing abnormal lipid levels, e.g., abnormally low HDL-C level, or a disease or disorder associated with abnormal lipid levels, e.g., a vascular or metabolic disease or disorder.

In one embodiment, the disease or condition is characterized by aberrant protein activity, such as aberrant protein level, which can result from aberrant expression of a gene listed in Tables 1-5. Accordingly, the invention provides methods for predicting abnormal lipid levels, e.g., abnormally low HDL-C level.

The invention also provides a method of identifying subjects which are at increased risk of developing abnormal lipid levels, e.g., abnormally low HDL-C level, or a disease or disorder associated with abnormal lipid levels, e.g., a vascular or metabolic disease or disorder, wherein the method comprises the steps of i) identifying in DNA from a subject at least one sequence polymorphism, as compared with the reference sequence, in a gene listed in Tables 1-5; and ii) identifying the subject based on the identified polymorphism.

In another embodiment, the invention provides a kit for amplifying and/or for determining the molecular structure of at least a portion of a gene listed in Tables 1-5, comprising a probe or primer capable of hybridizing to a gene listed in Tables 1-5 and instructions for use. In a preferred embodiment, determining the molecular structure of a region of a gene listed in Tables 1-5 comprises determining the identity of the allelic variant of the polymorphic region. Determining the molecular structure of at least a portion of a gene listed in Tables 1-5 can comprise determining the identity of at least one nucleotide or determining the nucleotide composition, e.g., the nucleotide sequence of a gene listed in Tables 1-5.

A kit of the invention can be used, e.g., for determining whether a subject is or is not at risk of developing a disease associated with a specific allelic variant of a polymorphic region of a gene listed in Tables 1-5. In a preferred embodiment, the invention provides a kit for determining whether a subject is or is not at risk of developing abnormal lipid levels, e.g., abnormally low HDL-C level, or a disease or disorder associated with abnormal lipid levels, e.g., a vascular or metabolic disease or disorder. The kit of the invention can also be used in selecting the appropriate clinical course of treatment for a subject. Thus, determining the allelic variants of polymorphic regions within a gene listed in Tables 1-5 in a subject can be useful in predicting how a subject will respond to a specific drug, e.g., a drug for treating a disease or disorder associated with a polymorphism of a gene listed in Tables 1-5, e.g., abnormal lipid levels, e.g., abnormally low HDL-C level, or a disease or disorder associated with abnormal lipid levels, e.g., a vascular or metabolic disease or disorder.

In a further embodiment, the invention provides a method for treating a subject having a disease or condition associated with a specific allelic variant of a polymorphic region of a gene listed in Tables 1-5. In one embodiment, the method comprises the steps of (a) determining the identity of the allelic variant; and (b) administering to the subject a clinical course of therapy that compensates for the effect of the specific allelic variant e.g., treatment with medications, lifestyle changes, and any combination thereof. In one embodiment, the clinical course of therapy is administration of an agent or modulator which modulates, e.g., agonizes or antagonizes, nucleic acid expression or protein levels. In a preferred embodiment, the modulator is selected from the group consisting of a nucleic acid, a ribozyme, an antisense nucleic acid molecule, a protein or polypeptide, an antibody, a peptidomimetic, or a small molecule.

In a preferred embodiment, the specific allelic variant is a mutation. The mutation can be located, e.g., in a 5′ upstream regulatory element, a 3′ regulatory element, an intron, or an exon of the gene.

Additionally, the invention provides a method of identifying a subject who is susceptible to abnormal lipid levels, e.g., abnormally low HDL-C level, which method comprises the steps of i) providing a nucleic acid sample from a subject; and ii) detecting in the nucleic acid sample one or more allelic variants of a gene listed in Tables 1-5 which correlate with the vascular disorder with a P value less than or equal to 0.05, the existence of the polymorphism being indicative of susceptibility to abnormal lipid levels, e.g., abnormally low HDL-C level.

In another aspect, the invention provides methods for predicting the effect of hormone replacement therapy (HRT) on the HDL-C level in a female subject, e.g., a postmenopausal female subject, comprising identifying one or more allelic variants of a gene listed in Tables 1-5 which are associated with abnormally low HDL-C level in females, thereby predicting the effect of hormone replacement therapy on the HDL-C level in the subject. In particular, the presence of AT3_{—}1 AG or AA, F2_{—}1 TT, ITGB3_{—}4 TC, LIPC_{—}1 AG or AA, LRP1_{—}1 GT or TT, PPARG_{—}1 CC, PRCP_{—}1 CC, THBS4_{—}1 GG or GC, or the complements thereof, indicates the effect of hormone replacement therapy in a female subject to be a decrease in HDL-C level. The presence of COL5A2_{—}1 GG, CD14_{—}1 CT, VWF_{—}2 AG, and ITGB3_{—}4 TC, in combination, or the complements thereof, also indicate the effect of hormone replacement therapy in a female subject to be a decrease in HDL-C level.

The invention further provides forensic methods based on detection of polymorphisms within the genes listed in Tables 1-5.

Other features and advantages of the invention will be apparent from the following detailed description and claims.

### DETAILED DESCRIPTION OF THE INVENTION

The present invention is based, at least in part, on the identification of polymorphic regions within several genes which are associated with abnormal lipoprotein levels, e.g., low HDL-C levels (see Tables 1-5, below). Decreased HDL-C levels is a well-known risk factor for the development of vascular diseases and disorders, e.g., CAD and MI and metabolic diseases and disorders, e.g. diabetes and obesity. Accordingly, SNPs in these genes, as identified herein, can be utilized to predict the risk, in a subject, of developing abnormal lipid levels, e.g., abnormally low HDL-C level, or a disease or disorder associated with abnormal lipid levels, e.g., a vascular or metabolic disease or disorder. In particular, a subject having a specific allele listed in Table 5 is at an increased risk of having or developing abnormal lipid levels, e.g., low HDL-C levels, or a disease or disorder associated with abnormal lipid levels, e.g., a vascular or metabolic disease or disorder.

Furthermore, these polymorphisms may be useful in predicting the effect of hormone replacement therapy (HRT) on lipid levels in female subjects, e.g., postmenopausal female subjects, and therefore the risk for developing a vascular or metabolic disease or disorder.

Table 1, below, lists the SNPs which are associated with HDL-C levels, and comprises the SNPs set forth in Tables 2, 3, 4 and 5. Table 2 lists SNPs which are associated with HDL-C level in both male and female subjects. Table 3 lists gender specific SNPs which are associated with HDL-C in either males or females. The present invention also includes those polymorphisms in LD with the SNPs in Tables 2 and 3, as shown, for example, in Table 4. Table 5 lists the specific alleles of the SNPs listed in Tables 2, 3, and 4 which indicate that a subject is more likely to have, develop, or be at a higher than normal risk of developing abnormal lipid levels, e.g., low HDL-C levels, or a disease or disorder associated with abnormal lipid levels, e.g., a vascular disease or disorder or a metabolic disease or disorder.

TABLE 1
Sequence and position of SNPs associated with HDL-C.

| | | | 4 | | | | | 9 |
|---|---|---|---|---|---|---|---|---|
| 1 | 2 | 3 | Mutation | 5 | 6 | 7 | 8 | Genbank Accession |
| gene | SNP ID No. | Var freq | type | Ref NT | Var NT | Flanking sequence | SEQ ID NO | #, NT position |
| APOA1 | APOA1_{—} | .17 | Promoter | C | T | TGATAAGCCCAGCCC[CT] | SEQ ID NO:1 | GI: 5764724 |
| | | | | | | GGCCCTGTTGCTGCT | | (SEQ ID NO:27) |
| | | | | | | | | nt. 123408 |
| AT3 | AT3_1 | .36 | Silent | G | A | GAGCCTGGCCAAGGT[GA] | SEQ ID NO:2 | GI: 9931231 |
| | | | | | | GAGAAGGAACTCACC | | (SEQ ID NO:28) |
| | | | | | | | | nt. 77082 |
| CD14 | CD14_1 | .49 | Promoter | C | T | TCCTTCCTGTTACGG[CT] | SEQ ID NO:3 | GI: 400457 |
| | | | | | | CCCCCTCCCTGAAAC | | (SEQ ID NO:29) |
| | | | | | | | | nt. 2232 |
| COL5A2 | COL5A2_1 | .18 | Silent | G | A | CCCAACGGGCTCTCC[GA] | SEQ ID NO:4 | GI: 179697 |
| | | | | | | GGTACCTCTGGTCCT | | (SEQ ID NO:30) |
| | | | | | | | | nt. 1449 |
| ECE1 | ECE1_1 | .28 | Silent | G | A | CTCTTACCATCTGTC[GA] | SEQ ID NO:5 | GI: 14530800 |
| | | | | | | GTGGTGTTGATGAGA | | (SEQ ID NO:31) |
| | | | | | | | | nt. 75221 |
| EDNRB | EDNRB_1 | .40 | Silent | G | A | AAAAGATTGGTGGCT[GA] | SEQ ID NO:6 | GI: 2285955 |
| | | | | | | TTCAGTTTCTATTTC | | (SEQ ID NO:32) |
| | | | | | | | | nt 1064 |
| F2 | F2_1 | .12 | Mis (TM) | C | T | CCGACAGCAGCACCA[CT] | SEQ ID NO:7 | GI: 261694 |
| | | | | | | GGGACCCTGGTGCTA | | (SEQ ID NO:33) |
| | | | | | | | | nt. 42 |
| | | | | | | | | AA 165 |
| F2 | F2_3 | .06 | Non Coding | G | A | CTGCCTCCTGTACCC[GA] | SEQ ID NO:8 | GI: 558069 |
| | | | | | | CCCTGGGACAAGAAC | | (SEQ ID NO:34) |
| | | | | | | | | nt. 15419 |
| FABP3 | FABP3_1 | .04 | Mis (KR) | T | C | AAGGTGCTGTGTGTT[TC] | SEQ ID NO:9 | GI: 17902903 |
| | | | | | | TTAGGGTGAGAATGT | | (SEQ ID NO:35) |
| | | | | | | | | nt. 120178 |
| | | | | | | | | AA 207 |
| GBE1 | GBE1_1 | .03 | Mis (TA) | A | G | AACATGAGTGTCCTG[AG] | SEQ ID NO:10 | GI: 4557618 |
| | | | | | | CTCCTTTTACTCCAG | | (SEQ ID NO:36) |
| | | | | | | | | nt. 1597 |
| | | | | | | | | AA 506 |
| ITGB3 | ITGB3_2 | .29 | Silent | T | C | CTCCCGGGGGCTGCA[TC] | SEQ ID NO:11 | GI: 17488650 |
| | | | | | | TCGTCCTGCTGGGAA | | (SEQ ID NO:37) |
| | | | | | | | | nt: 98031 |
| ITGB3 | ITGB3_4 | .30 | Silent | C | T | TGCAGACGGGCTGAC[CT] | SEQ ID NO:12 | GI: 17488650 |
| | | | | | | CTCCCGGGGGCTGCA | | nt: 98019 |
| LIPC | LIPC_1 | .04 | Mis (VM) | G | A | CCTCAGGTGGACGGC[GA] | SEQ ID NO:13 | GI: 13374652 |
| | | | | | | TGCTAGAAAACTGGA | | (SEQ ID NO:38) |
| | | | | | | | | nt. 86244 |
| | | | | | | | | AA 95 |
| LIPC | LIPC_5 | .22 | Promoter | G | A | ACACAGTAGCTTTAA[GA] | SEQ ID NO:14 | GI: 187155 |
| | | | | | | TTGATTAATTTGGAA | | (SEQ ID NO:39) |
| | | | | | | | | nt. 1308 |
| LRP1 | LRP1_1 | .08 | Mis (VL) | G | T | CCCAACGGCATCTCA[GT] | SEQ ID NO:15 | GI: 3493562 |
| | | | | | | TGGACTACCAGGATG | | (SEQ ID NO:40) |
| | | | | | | | | nt. 1429 |
| LRP1 | LRP1_3 | .31 | Silent | C | T | CTTCCGGCTGMGGA[CT]G | SEQ ID NO:16 | GI: 3493567 |
| | | | | | | ACGGCCGGACGTGT | | (SEQ ID NO:41) |
| | | | | | | | | nt. 201 |
| LRP1 | LRP1_5 | .31 | Silent | C | T | TGAGGGCGAGCTCTG[CT] | SEQ ID NO:17 | GI: 3493558 |
| | | | | | | GGTGAGGCCTGGTCC | | (SEQ ID NO:42) |
| | | | | | | | | nt. 1209 |
| LRPAP1 | LRPAP1_1 | .10 | Silent | T | C | CTACAGCACTGAGGC[TC] | SEQ ID NO:18 | GI: 17977890 |
| | | | | | | GAGTTCGAGGAGCCC | | (SEQ ID NO:43) |
| | | | | | | | | nt. 51697 |
| MTHFR | MTHFR_1 | .32 | Mis | C | T | GATTTCATCATCA[CT]GC | SEQ ID NO:19 | GI: 4336810 |
| | | | (AV) | | | AGCTTTTCTTTGA | | (SEQ ID NO:44) |
| | | | | | | | | nt. 144 |
| PAI2 | PAI2_1 | .22 | Mis | G | C | TAGTTTTAGGGTGAG[GC] | SEQ ID NO:20 | GI: 6705901 |
| | | | (SC) | | | AAAATCTGCCGAAAA | | (SEQ ID NO:45) |
| | | | | | | | | nt. 164736 |
| PAI2 | PAI2_2 | .22 | Mis | G | C | GAAAAATAAAATGCA[GC] | SEQ ID NO:21 | GI: 6705901 |
| | | | (NK) | | | TTGGTTATCTTATGC | | nt. 164762 |
| PAI2 | PAI2_4 | .21 | Non | G | C | ATCTAGAAGCAAAGA[GC] | SEQ ID NO:22 | GI: 6705901 |
| | | | Coding | | | AGGAAGAAAAAAACA | | nt.176609 |
| PPARG | PPARG_1 | .12 | Mis | G | C | AGGAATCGCTTTCTG[GC] | SEQ ID NO:23 | GI: 13384351 |
| | | | (PA) | | | GTCAATAGGAGAATC | | (SEQ ID NO:46) |
| | | | | | | | | nt. 145136 |
| PRCP | PRCP_1 | .03 | Mis | G | C | GTGACTGCAACCAGA[GC] | SEQ ID NO:24 | GI: 12381909 |
| | | | (TS) | | | TGTCTGTGATATCCT | | (SEQ ID NO:47) |
| | | | | | | | | nt. 63956 |
| THBS4 | THBS4_1 | .20 | Mis | G | C | GAGTGTCGAAATGGA[GC] | SEQ ID NO:25 | GI: 14916146 |
| | | | (AP) | | | CGTGCGTTCCCAACT | | (SEQ ID NO:48) |
| | | | | | | | | nt. 105290 |
| VWF | VWF_2 | .12 | Silent | C | A | ACAGTCATTGGTGGC[GA] | SEQ ID NO:26 | GI: 4827300 |
| | | | | | | GTTGAGGCCAAGTAC | | (SEQ ID NO:49) |
| | | | | | | | | nt.18466 |

The polymorphisms of the present invention are single nucleotide polymorphisms (SNPs) at a specific nucleotide residue within the genes identified in Table 1. Each of these genes has at least two alleles, referred to herein as the reference allele and the variant allele. The reference allele (i.e., the consensus sequence, or wild type allele) has been designated based on it's frequency in a general U.S. Caucasian population sample. Nucleotide sequences in GenBank™ may correspond to either allele and correspond to the nucleotide sequence of the molecule which has been deposited in GenBank™ and given a specific Accession Number (e.g., GI 5764724 (SEQ ID NO: 27), the reference sequence for the APOA1 gene). As used herein, each reference sequence is identified by a “GI” GenBank™ Accession Number and a SEQ ID NO. The variant allele differs from the reference allele by at least one nucleotide at the site identified in Table 1. The present invention thus relates to nucleotides comprising either the reference allele or the variant allele of the genes identified in Table 1, and/or complements thereof, which are associated with low HDL-C. These alleles may be used in combination with each other or in combination with other SNPs to predict the risk of abnormal lipid levels, e.g., abnormally low HDL-C level, or a disease or disorder associated with abnormal lipid levels, e.g., a vascular or metabolic disease or disorder.

It is understood that the invention is not limited by these exemplified reference sequences, as variants of these sequences which differ at locations other than the SNP sites identified herein can also be utilized as reference sequences. The skilled artisan can readily determine the SNP sites in these other reference sequences which correspond to the SNP site identified herein by aligning the sequence of interest with the reference sequences specifically disclosed herein. Programs for performing such alignments are commercially available. For example, the ALIGN program in the GCG software package can be used, utilizing a PAM120 weight residue table, a gap length penalty of 12 and a gap penalty of 4, for example. Moreover, the reference sequences exemplified herein may or may not represent the coding strands of the genes described herein. One of skill in the art would readily be able to identify the coding strands for each of the genes described herein. Accordingly, both the coding strands and the non-coding strands of the polymorphic regions and SNPs described herein are understood to be included in the instant invention.

Cases which were used to identify associations between abnormal lipid levels and SNPs were drawn from the GeneQuest study, a collection of families with premature coronary artery disease. Subjects in the GeneQuest study all had premature CAD identified at one of 15 participating medical centers, fulfilling the criteria of either myocardial infarction, surgical or percutaneous revascularization, or a significant coronary artery lesion diagnosed before age 45 in males or age 50 in females and having a living sibling who met the same criteria. For this study, one individual per family was selected for genotyping. The final sample was comprised of 352 Caucasian individuals with a personal and family history of premature CAD.

Most of the allelic variants of the present invention were identified through denaturing high performance liquid chromatography (DHPLC) analysis, variant detector arrays (Affymetrix™), the polymerase chain reaction (PCR), and/or single stranded conformation polymorphism (SSCP) analysis using PCR primers complementary to intronic sequences surrounding each of the exons, 3′ UTR, and 5′ upstream regulatory element sequences of the genes. The polymorphic regions of the present invention have been identified in the genes by analyzing the DNA of cell lines derived from an ethnically diverse population by methods described in Cargill, et al. (1999 Nature Genetics 22:231-238).

The presence of polymorphisms in several genes were identified. The preferred polymorphisms of the invention are listed in Table 1. Table 1 contains a “SNP ID No.” in column 2, which is used herein to identify each of the variants. Each SNP is also designated by a SEQ ID NO in column 8 (SEQ ID NOs.: 1-26). The SEQ ID NO. contains the variant nucleotide as well as the 15 nucleotides flanking the polymorphic nucleotide residue (i.e., 15 nucleotides 5′ of the polymorphism and 15 nucleotides 3′ of the polymorphism), shown in column 7. The variant nucleotide is indicated by the second nucleotide contained within the brackets, e.g., a C→T change is indicated as [C/T]. Column 4 describes the type of variant, e.g., either non-coding, missense, silent, or promoter.

The polymorphisms are identified based on a change in the nucleotide sequence from a consensus sequence, or the “reference sequence.” To identify the location of the polymorphisms of the present invention, a specific nucleotide residue in a reference sequence is listed for the polymorphism, where nucleotide residue number 1 is the first (i.e., 5′) nucleotide in each reference sequence. Column 9 of Table 1 lists the reference sequence and polymorphic nucleotide residue for the polymorphisms.

Significant associations (p≦0.05) were found in both males and females between HDL-C levels and SNPs in nine genes as set forth in Table 2, below.

TABLE 2
Mean HDL-C levels by genotype in GeneQuest population for significant
associations.

| SNP | Genotype | N | Mean HDL-C (SD) | p-value |
|---|---|---|---|---|
| APOA1_1 | CC | 206 | 37.7 (9.6) | .01 |
| | TC/TT | 102 | 41.2 (13.3) | |
| CD14_1 | TT | 84 | 41.5 (11.1) | .002 |
| | CT | 149 | 36.5 (9.9) | |
| | CC | 81 | 39.8 (11.7) | |
| COL5A2_1 | GG | 180 | 39.5 (10.2) | .0006 |
| | AG | 84 | 42.4 (11.3) | |
| | AA | 8 | 53.1 (14.7) | |
| EDNRB_1 | GG | 125 | 42.0 (11.7) | .005 |
| | AG | 160 | 37.9 (10.7) | |
| | AA | 38 | 37.8 (8.1) | |
| FABP3_1 | TT | 314 | 39.5 (11.1) | .01 |
| | CT | 25 | 33.7 (8.0) | |
| GBE1_1 | AA | 315 | 39.5 (10.9) | .01 |
| | AG/GG | 24 | 33.8 (10.4) | |
| LIPC_5 | GG | 184 | 38.3 (10.5) | .03 |
| | AG | 109 | 41.1 (12.5) | |
| | AA | 23 | 35.4 (8.3) | |
| MTHFR_1 | CC | 146 | 37.2 (10.1) | .05 |
| | CT | 112 | 39.4 (11.6) | |
| | TT | 45 | 41.4 (11.4) | |
| VWF_2 | GG | 253 | 38.4 (10.8) | .04 |
| | GA | 63 | 41.5 (12.0) | |
| | AA | | | |

SD = Standard deviation.

As shown in Table 2, a subject (either male or female) having the APOA_{—}1 CC genotype, the CD14_{—}1 CT genotype, the COL5A2_{—}1 GG genotype, the EDNRB_{—}1 AG or AA genotype, the FABP3_{—}1 CT genotype, the GBE1_{—}1 AG or GG genotype, the LIPC_{—}5 AA genotype, the MTHFR_{—}1 CC genotype, or the VWF_{—}2 GG genotype is more likely to have or to develop abnormal lipid levels, e.g., an abnormally low HDL-C level.

Associations between HDL-C and variants in ten additional genes were found when males and females were analyzed separately (see Table 3, below). These SNPs, identified through significant SNP by sex interaction, usually conferred the opposite effect in males and females. However, the effect in females was typically stronger, resulting in significant associations (p≦0.05).

TABLE 3
Gender-specific mean HDL-C by genotype for significant interactions.

| | Intx P | Geno- | | Mean (SD) | P-value | | Mean (SD) | P-value |
|---|---|---|---|---|---|---|---|---|
| SNP | value | type | N | males | males | N | females | females |
| AT3_1 | .02 | GG | 86 | 36.6 (9.7) | .54 | 40 | 45.9 (11.7) | .03 |
| | | AG | 96 | 38.1 (10.7) | | 38 | 39.5 (10.7) | |
| | | AA | 28 | 36.3 (12.0) | | 14 | 39.9 (10.0) | |
| ECEl_1 | .03 | GG | 104 | 37.0 (10.6) | .12 | 50 | 42.8 (11.2) | .19 |
| | | AG | 86 | 36.8 (10.1) | | 35 | 43.0 (11.7) | |
| | | AA | 19 | 42.3 (13.4) | | 4 | 32.3 (11.4) | |
| F2_1 | .008 | CC | 173 | 37.6 (10.5) | .24 | 77 | 41.1 (10.6) | .05 |
| | | CT | 44 | 35.0 (9.6) | | 16 | 48.6 (14.5) | |
| | | TT | 5 | 41.0 (8.4) | | 3 | 38.0 (5.3) | |
| ITGB3_4 | .02 | CC | 98 | 37.1 (10.7) | .72 | 46 | 42.2 (10.7) | .005 |
| | | TC | 77 | 37.9 (10.4) | | 28 | 38.5 (8.1) | |
| | | TT | 24 | 39.1 (12.9) | | 55 | 55.2 (17.3) | |
| LIPC_1 | .03 | GG | 203 | 37.4 (10.6) | .84 | 91 | 43.3 (11.3) | .02 |
| | | AG/AA | 20 | 37.8 (10.5) | | 6 | 32.3 (8.6) | |
| LRP1_1 | .01 | GG | 185 | 37.5 (10.7) | .50 | 78 | 44.1 (11.2) | .02 |
| | | GT/TT | 43 | 38.7 (10.9) | | 20 | 37.3 (10.7) | |
| LRP1_3 | .03 | CC | 79 | 37.3 (11.1) | .05 | 33 | 43.8 (11.5) | .19 |
| | | CT | 99 | 36.5 (10.1) | | 51 | 42.3 (12.0) | |
| | | TT | 17 | 43.4 (12.3) | | 4 | 32.5 (4.2) | |
| LRPAP1_1 | .05 | TT | 180 | 37.3 (10.4) | .17 | 73 | 43.9 (11.7) | .16 |
| | | CT/CC | 40 | 39.8 (10.7) | | 19 | 39.7 (10.8) | |
| PAI2_4 | .02 | CC | 113 | 38.7 (10.9) | .05 | 55 | 42.8 (12.1) | .13 |
| | | CG | 62 | 41.2 (10.0) | | 28 | 43.9 (9.3) | |
| | | GG | 9 | 32.6 (10.6) | | 45 | 54.8 (31.6) | |
| PPARG_1 | .007 | CC | 184 | 38.1 (10.8) | .05 | 83 | 41.2 (11.1) | .03 |
| | | CG | 46 | 34.9 (8.8) | | 16 | 48.4 (11.1) | |
| | | GG | 6 | 44.0 (10.0) | | 1 | 57.0 | |
| PRCP_1 | .003 | CC | 221 | 38.0 (10.7) | .06 | 96 | 41.8 (11.3) | .02 |
| | | CG/GG | 19 | 33.3 (6.1) | | 5 | 53.8 (2.2) | |
| THBS4_1 | .006 | GG | 134 | 36.8 (10.6) | .42 | 58 | 43.8 (11.4) | .02 |
| | | CG | 86 | 38.5 (9.9) | | 35 | 38.7 (9.9) | |
| | | CC | 13 | 35.5 (10.7) | | 7 | 50.4 (13.1) | |

SD = Standard deviation.

As shown in Table 3, the AT3_{—}1, F2_{—}1, ITGB3_{—}4, LIPC_{—}1, LRP1_{—}1, PRCP_{—}1, and THBS4_{—}1 SNPs had a significant association with HDL-C level in females. The LRP1_{—}3, PAI2_{—}4, and PPARG_{—}1 SNPs had a significant association with HDL-C level in males.

A female subject having the AT3_{—}1 AG or AA genotype, the F2_{—}1 TT genotype, the ITGB3_{—}4 TC genotype, the LIPC_{—}1 AG or AA genotype, the LRP1_{—}1 GT or TT genotype, the PPARG_{—}1 CC genotype, the PRCP_{—}1 CC genotype, or the THBS4_{—}1 GG or GC genotype is more likely to have or to develop abnormal lipid levels, e.g., abnormally low HDL-C levels.

A male subject having the LRP1_{—}3 CC or CT genotype, the PAI2_{—}4 GG genotype, or the PPARG_{—}1 CG genotype is more likely to have or to develop abnormal lipid levels, e.g., abnormally low HDL-C levels.

For some genes multiple SNPs were typed. In some cases, SNPs in a gene were highly correlated, or in linkage disequilibrium with each other, and yet not all of these SNPs showed significant (p≦0.05) associations with HDL-C. The term “linkage disequilibrium,” also referred to herein as “LD,” refers to a greater than random association between specific alleles at two marker loci within a particular population. A SNP in linkage disequilibrium with another SNP which shows an association may be considered as a marker for the SNP with an association, and, therefore, a risk factor (albeit not independent of the associated SNP). Accordingly, if linkage disequilibrium exists between two markers, or SNPs, then the genotypic information at one marker, or SNP, can be used to make probabilistic predictions about the genotype of the second marker.

Table 4, below, shows SNPs which are in linkage disequilibrium with certain SNPs of the invention. These SNPs, therefore, may also be used as markers for abnormal lipid levels, e.g., abnormally low HDL-C levels.

TABLE 4
SNPs in linkage disequilibrium with associated SNPs from Tables 2 and 3.

| gene | SNP pair | D′ | P |
|---|---|---|---|
| F2 | F2_1 | −.77 | .008 |
| | F2_3 | | |
| ITGB3 | ITGB3_2 | .99 | <.0001 |
| | ITGB3_4 | | |
| LRP1 | LRP1_3 | .99 | <.0001 |
| | LRP1_5 | | |
| PAI2 | PAI2_1 | 1.0 | <.0001 |
| | PAI2_2 | | |
| PAI2 | PAI2_1 | .80 | <.0001 |
| | PAI2_4 | | |
| PAI2 | PAI2_2 | .80 | <.0001 |
| | PAI2_4 | | |

For example, in the LRP1 gene, the LRP1_{—}3 SNP and the LRP1_{—}5 SNP are in LD (D′=0.99; p=<0.0001). LRP1_{—}3 shows a significant association with abnormal lipid level in males (p<0.05; see Table 3). Although LRP1_{—}5 does not show a significant association with abnormal lipid level in the population tested, it may be used as a marker for abnormal lipid level because it is in LD with LRP1_{—}3. For two genes where multiple SNPs were typed, more than one SNP showed a statistically significant association with HDL-C. In the LIPC gene, both the LIPC_{—}1 and LIPC_{—}5 SNPs were associated with HDL-C. These two SNPs are not in linkage disequilibrium (D′=0.10, p=0.37). Therefore, they represent independent risk factors. Similarly, in the LRP1 gene, two SNPs were significantly associated with HDL-C, LRP1_{—}1 and LRP1_{—}3. These SNPs are not in linkage disequilibrium (D′=−0.13, p=0.49) and therefore represent independent associations.

Results from a multivariate analysis (see Table 6 and Table 7, in the Example section) revealed that different combinations of genes may influence HDL-C levels in males and females. In females, five genes were independently associated with HDL-C including COL5A2, CD14, F2, VWF and ITGB3. In our population, the combination of these five SNPs account for approximately 65% of the variability in HDL-C (overall model p<0.0001).

Female subjects having or at risk for developing the lowest levels of HDL-C are those with the following combination of genotypes: COL5A2_{—}1 GG, CD14_{—}1 CT, VWF_{—}2 GA and ITGB3_{—}4 TC. This combination is estimated to have a frequency of approximately 3% in a general U.S. Caucasian population.

In males, a different combination of three genes was identified. COL5A2, CD14, and FABP3 were independently associated with HDL-C and together account for approximately 21% of the variation in HDL-C in males (overall model p<0.0001).

Male subjects having or at risk for developing the lowest levels of HDL-C are those with the following combination of genotypes: COL5A2_{—}1 GG, CD14_{—}1 CT or CC, and FABP3_{—}1 CT. This combination is estimated to have a frequency of approximately 4% in a general U.S. Caucasian population.

Accordingly, specific alleles which indicate that a subject is more likely to have or to be at a higher than normal risk of developing abnormal lipid levels, e.g., low HDL-C levels, or a disease or disorder associated with abnormal lipid levels, e.g., a vascular disease or disorder or a metabolic disease or disorder, are set forth in Table 5, below.

TABLE 5

| | | |
|---|---|---|
| Gene | SNP | Alleles |

Male or Female:

| | | |
|---|---|---|
| APOA1 | APOA_1 | CC |
| CD14 | CD14_1 | CT |
| COL5A2 | COL5A2_1 | GG |
| EDNRB | EDNRB_1 | AG or AA |
| FABP3 | FABP3_1 | CT |
| GBE1 | GBE1_1 | AG or GG |
| LIPC | LIPC_5 | AA |
| MTHFR | MTHFR_1 | CC |
| VFW | VWF_2 | GG |

Female:

| | | |
|---|---|---|
| AT3 | AT3_1 | AG or AA |
| F2 | F2_1 | TT |
| ITGB3 | ITGB3_4 | TC |
| LIPC | LIPC_1 | AG or AA |
| LRP1 | LRP1_1 | GT or TT |
| PPARG | PPARG_1 | CC |
| PRCP | PRCP_1 | CC |
| THBS4 | THBS4_1 | GG or GC |
| COL5A2 | COL5A2_1 | GG |
| CD14 | CD14_1 | CT |
| VWF | VWF_2 | GA |
| ITGB3 | ITGB3_4 | TC |
| | (in combination) | |

Male:

| | | |
|---|---|---|
| LRP1 | LRP1_3 | CC or CT |
| PAI | PAI2_4 | GG |
| PPARG | PPARG_1 | CG |
| COL5A2 | COL5A2_1 | GG |
| CD14 | CD14_1 | CT or CC |
| FABP3 | FABP3_1 (in combination) | CT |

The invention further relates to nucleotides comprising portions of the variant alleles and/or portions of complements of the variant alleles which comprise the site of the polymorphism and are at least 5 nucleotides or basepairs in length. Portions can be, for example, 5-10, 5-15, 10-20, 2-25, 10-30, 10-50 or 10-100 bases or basepairs long. For example, a portion of a variant allele which is 31 nucleotides or basepairs in length includes the polymorphism (i.e., the nucleotide(s) which differ from the reference allele at that site) and thirty additional nucleotides or basepairs which flank the site in the variant allele. These additional nucleotides and basepairs can be on one or both sides of the polymorphism. The polymorphisms which are the subject of this invention are defined in Table 1 with respect to the reference sequence identified in Table 1.

The nucleic acid molecules of the invention can be double- or single-stranded. Accordingly, the invention further provides for the complementary nucleic acid strands comprising the polymorphisms listed in Tables 1-5.

The invention further provides allele-specific oligonucleotides that hybridize to a gene comprising a SNP or to the complement of the gene. Such oligonucleotides will hybridize to one polymorphic form of the nucleic acid molecules described herein but not to the other polymorphic form of the sequence. Thus such oligonucleotides can be used to determine the presence or absence of particular alleles of the polymorphic sequences described herein. These oligonucleotides can be probes or primers. Other aspects of the invention are described below or will be apparent to one of skill in the art in light of the present disclosure.

Description Of Genes Containing SNPs of the Present Invention

APOA1

Apolipoprotein A-I (APOA1) promotes cholesterol efflux from tissues to the liver for excretion. APOA1 is the major protein component of high density lipoprotein (HDL) in the plasma. Synthesized in the liver and small intestine, it consists of two identical chains of 77 amino acids; an 18-amino acid signal peptide is removed co-translationally and a 6-amino acid propeptide is cleaved post-translationally. Variation in the latter step, in addition to modifications leading to so-called isoforms, is responsible for some of the polymorphisms observed. APOA1 is a cofactor for lecithin cholesterolacyltransferase (LCAT) which is responsible for the formation of most plasma cholesteryl esters. The APOA1, APOC3 and APOA4 genes are closely linked in both rat and human genomes. The A-I and A-IV genes are transcribed from the same strand, while the C-III gene is transcribed convergently in relation to A-I. Defects in the apolipoprotein A-1 gene are associated with HDL deficiency and Tangier disease.

AT3

Antithrombin III (AT3) is the sole blood component through which heparin exerts its anticoagulant effect. In persons with AT3 deficiency the effect may lead to recurrent thrombosis despite heparin therapy.

CD14

CD14 is a single-copy gene encoding 2 protein forms: a 50- to 55-kD lycosylphosphatidylinositol-anchored membrane protein (mCD14) and a monocyte or liver-derived soluble serum protein (sCD14) that lacks the anchor. Both molecules are critical for lipopolysaccharide (LPS)-dependent signal transduction, and sCD14 confers LPS sensitivity to cells lacking mCD14. The expression profile of CD14, as well as its inclusion in the family of leucine-rich proteins and the chromosomal location of other receptor genes, supports the hypothesis that CD14 functions as a receptor Glycoprotein CD14 on the surface of human macrophages is important for the recognition and clearance of apoptotic cells. CD14 can also act as a receptor that binds bacterial LPS, triggering inflammatory responses.

COL5A2

Type V collagen (COL5A2) is a member of group I collagen (fibrillar forming collagen). This gene encodes an alpha chain for one of the low abundance fibrillar collagens. Fibrillar collagen molecules are trimers that can be composed of one or more types of alpha chains. Type V collagen is found in tissues containing type I collagen and appears to regulate the assembly of heterotypic fibers composed of both type I and type V collagen. This gene product is closely related to type XI collagen and it is possible that the collagen chains of types V and XI constitute a single collagen type with tissue-specific chain combinations. Mutations in this gene are associated with Ehlers-Danlos syndrome, types I and II. Two transcripts that differ in the length of the 3′UTR due to the use of alternative polyadenylation signals have been identified for this gene.

ECE1

Endothelin converting enzyme (ECE1) is a metalloprotease that regulates a peptide involved in vasoconstriction.

EDNRB

Endothelin receptor type B (EDNRB) is a G protein-coupled receptor which activates a phosphatidylinoitol-calcium second messenger system. Its ligand, endothelin, consists of a family of three potent vasoactive peptides: ET1, ET2, and ET3. Studies suggest that the multigenic disorder, Hirschsprung disease type 2, is due to mutation in endothelin receptor type B gene. A splice variant, named SVR, has been described; the sequence of the ETB-SVR receptor is identical to ETRB except for the intracellular C-terminal domain. While both splice variants bind ET 1, they exhibit different responses upon binding which suggests that they may be functionally distinct.

F2

Coagulation factor II (F2) is proteolytically cleaved to form thrombin in the first step of the coagulation cascade which ultimately results in the stemming of blood loss. F2 also plays a role in maintaining vascular integrity during development and postnatal life. Mutations in F2 lead to various forms of thrombosis and dysprothrombinemia.

FABP3

Fatty acid metabolism in mammalian cells depends on a flux of fatty acids, between the plasma membrane and mitochondria or peroxisomes for beta-oxidation, and between other cellular organelles for lipid synthesis. At least 3 different fatty acid-binding proteins (FABPs) play a role as transport vehicles of these hydrophobic compounds throughout the cytoplasm. Different FABPs have been demonstrated in the liver, heart, intestine, skeletal muscle, and brain.

GBE1

The glucan (1,4-alpha)-branching enzyme-1 (GBE 1) is required for sufficient glycogen accumulation. The alpha 1-6 branches of glycogen play an important role in increasing the solubility of the molecule and, consequently, in reducing the osmotic pressure within cells. Highest levels found in liver and muscle. GBE1 belongs to family 13 of glycosyl hydrolases, also known as the alpha-amylase family. Mutations in this gene cause a rare form of glycogenosis, glycogen storage disease IV.

ITGB3

The ITGB3 protein product is the integrin beta chain beta 3. Integrins are integral cell-surface proteins composed of an alpha chain and a beta chain. A given chain may combine with multiple partners resulting in different integrins. ITGB3 is found along with the alpha IIb chain in platelets. Integrins are known to participate in cell adhesion as well as cell-surface mediated signaling.

LIPC

LIPC encodes hepatic triglyceride lipase, which is expressed in liver. LIPC has the dual functions of triglyceride hydrolase and ligand/bridging factor for receptor-mediated lipoprotein uptake.

LRP1

LRP is identical to the alpha-2-macroglobulin receptor (A2MR). Like the mannose-6-phosphate receptor (147280), the A2MR/LRP molecule is probably bi-functional. LRP1 had been shown to function as a receptor for the uptake of apolipoprotein E-containing lipoprotein particles by neurons.

LRPAP1

Alpha-2-macroglobulin receptor-associated protein (LRPAP1) prevents premature binding of ligands to receptors. LRPAP1 also enables correct folding and export from the ER of alpha-2-macroglobulin receptor, and affects interactions between plasma membranes and basement membranes.

MTHFR

MTHFR is an enzyme that catalyzes the conversion of 5,10-methylenetetrahydrofolate to 5-methyltetrahydrofolate, a co-substrate for homocysteine remethylation to methionine, which is important in folate metabolism.

PAI2

The specific inhibitors of plasminogen activators have been classified into 4 immunologically distinct groups: PAI1 type PA inhibitor from endothelial cells; PAI2 type PA inhibitor from placenta, monocytes, and macrophages; urinary inhibitor; and protease-nexin-I. Plasminogen activator inhibitor-2 (PAI2) is also known as monocyte arg-serpin because it belongs to the superfamily of serine proteases in which the target specificity of each is determined by the amino acid residue located at its reactive center; i.e., met or val for elastase, leu for kinase, and arg for thrombin. PAI2 is thought to serve as a primary regulator of plasminogen activation in the extravascular compartment. High levels of PAI2 are found in keratinocytes, monocytes, and the human trophoblast, the latter suggesting a role in placental maintenance or in embryo development. The primarily intracellular distribution of PAI2 may also indicate a unique regulatory role in a protease-dependent cellular process such as apoptosis.

PPARG

The peroxisome proliferator-activated receptors (PPARs) are members of the nuclear hormone receptor subfamily of transcription factors. PPARs form heterodimers with retinoid X receptors (RXRs) and these heterodimers regulate transcription of various genes. There are 3 known subtypes of PPARs, PPAR-alpha, PPAR-delta, and PPAR-gamma. PPAR-gamma is believed to be involved in adipocyte differentiation. PPAR-gamma is induced in human monocytes following exposure to oxLDL and is expressed at high levels in the foam cells of atherosclerotic lesions. Endogenous PPAR-gamma ligands may be important regulators of gene expression during atherogenesis. In addition to lipid uptake, PPARG regulates a pathway of cholesterol efflux. There are 3 PPARG isoforms which differ at their 5-prime ends, each under the control of its own promoter. PPARG1 and PPARG3, however, give rise to the same protein, encoded by exons 1 through 6.

PRCP

Prolylcarboxypeptidase (PRCP) cleaves C-terminal amino acids linked to a penultimate proline, if proline has a protected amino group or is part of a peptide chain. Because angiotensins II and III have the same C terminus and are substrates for the enzyme, prolylcarboxypeptidase was named angiotensinase C. The protein comprises 451 amino acids.

THBS4

The thrombospondins are a family of extracellular calcium binding proteins involved in cell proliferation, adhesion, and migration. The human thrombospondin-4 (THBS4) gene has been isolated from a heart expression library. Electron microscopy indicated that it is composed of 5 subunits with globular domains at each end. THBS4 binds to heparin and calcium.

VWF

The glycoprotein encoded by this gene functions as both an antihemophilic factor carrier and a platelet-vessel wall mediator in the blood coagulation system. It is crucial to the hemostasis process. Mutations in this gene or deficiencies in this protein result in von Willebrand's disease.

Definitions

For convenience, the meaning of certain terms and phrases employed in the specification, examples, and appended claims are provided below.

The term “allele,” which is used interchangeably herein with “allelic variant” refers to alternative forms of a gene or portions thereof. Alleles occupy the same locus or position on homologous chromosomes. When a subject has two identical alleles of a gene, the subject is said to be homozygous for the gene or allele. When a subject has two different alleles of a gene, the subject is said to be heterozygous for the gene or allele. Alleles of a specific gene can differ from each other in a single nucleotide, or several nucleotides, and can include substitutions, deletions, and insertions of nucleotides. An allele of a gene can also be a form of a gene containing one or more mutations.

The term “allelic variant of a polymorphic region of a gene” refers to an alternative form of a gene having one of several possible nucleotide sequences found in that region of the gene in the population.

“Biological activity” or “bioactivity” or “activity” or “biological function”, which are used interchangeably, for the purposes herein, with respect to the molecules described herein, e.g., the polypeptides encoded by the genes listed in Tables 1-5, means an effector or antigenic function that is directly or indirectly performed by a polypeptide (whether in its native or denatured conformation), or by a fragment thereof. Biological activities include modulation of the development of atherosclerotic plaque leading to vascular disease and other biological activities, whether presently known or inherent, binding to a ligand, e.g., a lipid or lipoprotein, such as LDL or modified forms thereof, or HDL or modified forms thereof, or a biological activity of a polypeptide as described above. A bioactivity can be modulated by directly affecting a corresponding protein effected by, for example, changing the level of effector or substrate level. Alternatively, a bioactivity can be modulated by modulating the level of a protein, such as by modulating expression of the gene encoding the protein. Antigenic functions include possession of an epitope or antigenic site that is capable of cross-reacting with antibodies that bind a native or denatured polypeptide or fragment thereof.

Biologically active polypeptides include polypeptides having both an effector and antigenic function, or only one of such functions. Polypeptides include antagonist polypeptides and native polypeptides, provided that such antagonists include an epitope of a native polypeptide. An effector function of a polypeptide can be the ability to bind to a ligand.

As used herein the term “bioactive fragment of a protein” refers to a fragment of a full-length protein, wherein the fragment specifically mimics or antagonizes the activity of a wild-type protein. The bioactive fragment preferably is a fragment capable of binding to a second molecule, such as a ligand.

The term “an aberrant activity” or “abnormal activity”, as applied to an activity of a protein encoded by a gene listed in Tables 1-5, refers to an activity which differs from the activity of the normal or reference protein or which differs from the activity of the protein in a healthy subject, e.g., a subject not afflicted with a disease associated with an allelic variant described herein. An activity of a protein can be aberrant because it is stronger than the activity of its wild-type counterpart. Alternatively, an activity of a protein can be aberrant because it is weaker or absent relative to the activity of its normal or reference counterpart. An aberrant activity can also be a change in reactivity. For example an aberrant protein can interact with a different protein or ligand relative to its normal or reference counterpart. A cell can also have aberrant activity due to overexpression or underexpression of the gene. Aberrant activity can result from a mutation in the gene, which results, e.g., in lower or higher binding affinity of a ligand to the protein encoded by the mutated gene. Aberrant activity can also result from an abnormal 5′ upstream regulatory element activity.

“Cells,” “host cells” or “recombinant host cells” are terms used interchangeably herein. It is understood that such terms refer not only to the particular cell but to the progeny or derivatives of such a cell. Because certain modifications may occur in succeeding generations due to either mutation or environmental influences, such progeny may not, in fact, be identical to the parent cell, but are still included within the scope of the term as used herein.

As used herein, the term “course of clinical therapy” refers to any chosen method to treat, prevent, or ameliorate abnormal lipid levels, e.g., abnormally low HDL-C levels, or a disease or disorder associated with abnormal lipid levels, e.g., a vascular or metabolic disease or disorder, symptoms thereof, or related diseases or disorders. Courses of clinical therapy include, but are not limited to, lifestyle changes (e.g., changes in diet, exercise, or environment), administration of medication, e.g., lipid modulating medication. Clinical course of therapy for treatment or prevention or amelioration of vascular disease in particular includes, for example, use of medical devices, such as, but not limited to, a defibrillator, a stent, a device used in coronary revascularization, a pacemaker, or any combination thereof, and surgical procedures such as percutaneous transluminal coronary balloon angioplasty (PTCA) or laser angioplasty, or other surgical intervention, such as, for example, coronary bypass grafting (CABG), or any combination thereof.

As used herein, the term “gene” or “recombinant gene” refers to a nucleic acid molecule comprising an open reading frame and including at least one exon and (optionally) an intron sequence. The term “intron” refers to a DNA sequence present in a given gene which is spliced out during mRNA maturation.

As used herein, the term “genetic profile” refers to the information obtained from identification of the specific allelic variants of a subject, e.g., the specific allelic variants of the SNPs identified in Tables 1-5. For example, genetic profile refers to the specific allelic variants of a subject within a gene identified in Tables 1-5. For example, one can determine a subject's APOA1 genetic profile by determining the identity of one or more of the nucleotides present at nucleotide residue 123408 of GI 5764724 (SEQ ID NO: 27, the APOA1 gene), or the complements thereof. The genetic profile of a particular disease can be ascertained through identification of the identity of allelic variants in one or more genes which are associated with the particular disease.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, though preferably less than 25% identity, with one of the sequences of the present invention.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical positions/total number of positions (e.g., overlapping positions)×100). In one embodiment the two sequences are the same length.

The determination of percent identity between two sequences can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the NBLAST and XBLAST programs of Altschul, et al. (1990) J. Mol. Biol. 215:403-410. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al. (1997) Nucleic Acids Res. 25:3389-3402. Alternatively, PSI-Blast can be used to perform an iterated search which detects distant relationships between molecules. When utilizing BLAST, Gapped BLAST, and PSI-Blast programs, the default parameters of the respective programs (e.g., XBLAST and NBLAST) can be used. Another preferred, non-limiting example of a mathematical algorithm utilized for the comparison of sequences is the algorithm of Myers and Miller, (1988) CABIOS 4:11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson and Lipman (1988) Proc. Natl. Acad. Sci. USA 85:2444-2448. When using the FASTA algorithm for comparing nucleotide or amino acid sequences, a PAM120 weight residue table can, for example, be used with a k-tuple value of 2.

The term “a homolog of a nucleic acid” refers to a nucleic acid having a nucleotide sequence having a certain degree of homology with the nucleotide sequence of the nucleic acid or complement thereof. For example, a homolog of a double stranded nucleic acid having SEQ ID NO: N is intended to include nucleic acids having a nucleotide sequence which has a certain degree of homology with SEQ ID NO: N or with the complement thereof. Preferred homologs of nucleic acids are capable of hybridizing to the nucleic acid or complement thereof.

The term “hybridization probe” or “primer” as used herein is intended to include oligonucleotides which hybridize bind in a base-specific manner to a complementary strand of a target nucleic acid. Such probes include peptide nucleic acids, and described in Nielsen et al, (1991) Science 254:1497-1500. Probes and primers can be any length suitable for specific hybridization to the target nucleic acid sequence. The most appropriate length of the probe and primer may vary depending on the hybridization method in which it is being used; for example, particular lengths may be more appropriate for use in microfabricated arrays, while other lengths may be more suitable for use in classical hybridization methods. Such optimizations are known to the skilled artisan. Suitable probes and primers can range form about 5 nucleotides to about 30 nucleotides in length. For example, probes and primers can be 5, 6, 8, 10, 12, 14, 16, 18, 20, 22, 24, 25, 26, 28 or 30 nucleotides in length. The probe or primer of the invention comprises a sequence that flanks and/or preferably overlaps, at least one polymorphic site occupied by any of the possible variant nucleotides. The nucleotide sequence of an overlapping probe or primer can correspond to the coding sequence of the allele or to the complement of the coding sequence of the allele.

A “disease or disorder associated with abnormal HDL-C level” or a “disease or disorder associated with abnormal lipid levels,” as used herein, includes any disease, disorder, or condition for which abnormal lipid levels, e.g., abnormal HDL-C levels, is a risk factor, e.g., a vascular or metabolic disease or disorder. The term “vascular disease or disorder” as used herein refers to any disease, disorder, or condition effecting the vascular system, including the heart and blood vessels. A vascular disease or disorder includes any disease, disorder or condition characterized by vascular dysfunction, including, for example, intravascular stenosis (narrowing) or occlusion (blockage), due to the development of atherosclerotic plaque and diseases and disorders resulting therefrom. Examples of vascular diseases and disorders include, without limitation, abnormal lipid metabolism, abnormal lipid level, abnormal HDL-C level, atherosclerosis, CAD, MI, ischemia, stroke, peripheral vascular diseases, venous thromboembolism and pulmonary embolism.

As used herein, the term “metabolic disease or disorder” includes a disorder, disease or condition which is caused or characterized by an abnormal metabolism, i.e., the chemical changes in living cells by which energy is provided for vital processes and activities in a subject. Metabolic diseases and disorders include diseases, disorders, or conditions associated with abnormal lipid levels, e.g., abnormal HDL-C level. Examples of metabolic diseases and disorders include obesity, diabetes, hyperphagia, endocrine abnormalities, triglyceride storage disease, Bardet-Biedl syndrome, Lawrence-Moon syndrome, Prader-Labhart-Willi syndrome, anorexia, and cachexia. Obesity is defined as a body mass index (BMI) of 30 kg/²m or more (National Institute of Health, Clinical Guidelines on the Identification, Evaluation, and Treatment of Overweight and Obesity in Adults (1998)). However, the present invention is also intended to include a disease, disorder, or condition that is characterized by a body mass index (BMI) of 25 kg/²m or more, 26 kg/²m or more, 27 kg/²m or more, 28 kg/²m or more, 29 kg/²m or more, 29.5 kg/²m or more, or 29.9 kg/²more, all of which are typically referred to as overweight (National Institute of Health, Clinical Guidelines on the Identification, Evaluation, and Treatment of Overweight and Obesity in Adults (1998)).

As used herein, the term “abnormally low” HDL-C level refers to an HDL-C level which is lower than the level generally accepted by one of skill in the art as being normal, e.g., lower than approximately 35 mg/dl in males or lower than approximately 40 mg/dl in females.

The term “interact” as used herein with respect to interaction between molecules, is meant to include detectable interactions between molecules, such as can be detected using, for example, a binding or hybridization assay. The term interact is also meant to include “binding” interactions between molecules. Interactions may be, for example, protein-protein, protein-nucleic acid, protein-small molecule or small molecule-nucleic acid in nature. The term “interaction” when used in the context of a statistical relationship or analysis, refers to a means for demonstrating the underlying effect of haplotypes, e.g., the combined effect of SNPs at different loci that are in LD.

The term “intronic sequence” or “intronic nucleotide sequence” refers to the nucleotide sequence of an intron or portion thereof.

The term “isolated” as used herein with respect to nucleic acids, such as DNA or RNA, refers to molecules separated from other DNAs or RNAs, respectively, that are present in the natural source of the macromolecule. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides.

The term “lipid” refers to a fat or fat-like substance that is insoluble in polar solvents such as water. The term “lipid” is intended to include true fats (e.g. esters of fatty acids and glycerol); lipids (phospholipids, cerebrosides, waxes); sterols (cholesterol, ergosterol) and lipoproteins (e.g. HDL, LDL and VLDL).

The term “linkage disequilibrium,” also referred to herein as “LD,” refers to a greater than random association between specific alleles at two marker loci within a particular population. In general, linkage disequilibrium decreases with an increase in physical distance. If linkage disequilibrium exists between two markers, then the genotypic information at one marker can be used to make probabilistic predictions about the genotype of the second marker.

The term “locus” refers to a specific position in a chromosome.

The term “modulation” as used herein refers to both upregulation, (i.e., activation or stimulation), for example by agonizing; and downregulation (i.e., inhibition or suppression), for example by antagonizing of a bioactivity (e.g. expression of a gene).

The term “molecular structure” of a gene or a portion thereof refers to the structure as defined by the nucleotide content (including deletions, substitutions, additions of one or more nucleotides), the nucleotide sequence, the state of methylation, and/or any other modification of the gene or portion thereof.

The term “mutated gene” refers to an allelic form of a gene that differs from the predominant form in a population. A mutated gene is capable of altering the phenotype of a subject having the mutated gene relative to a subject having the predominant form of the gene. If a subject must be homozygous for this mutation to have an altered phenotype, the mutation is said to be recessive. If one copy of the mutated gene is sufficient to alter the phenotype of the subject, the mutation is said to be dominant. If a subject has one copy of the mutated gene and has a phenotype that is intermediate between that of a homozygous and that of a heterozygous subject (for that gene), the mutation is said to be co-dominant.

As used herein, the term “nucleic acid” refers to polynucleotides such as deoxyribonucleic acid (DNA), and, where appropriate, ribonucleic acid (RNA). The term should also be understood to include, as equivalents, derivatives, variants and analogs of either RNA or DNA made from nucleotide analogs, and, as applicable to the embodiment being described, single (sense or antisense) and double-stranded polynucleotides. Deoxyribonucleotides include deoxyadenosine, deoxycytidine, deoxyguanosine, and deoxythymidine. For purposes of clarity, when referring herein to a nucleotide of a nucleic acid, which can be DNA or an RNA, the terms “adenine”, “cytidine”, “guanine”, and “thymidine” and/or “A”, “C”, “G”, and “T”, respectively, are used. It is understood that if the nucleic acid is RNA, a nucleotide having a uracil base is uridine.

The term “nucleotide sequence complementary to the nucleotide sequence set forth in SEQ ID NO: N” refers to the nucleotide sequence of the complementary strand of a nucleic acid strand having SEQ ID NO: N. The term “complementary strand” is used herein interchangeably with the term “complement.” The complement of a nucleic acid strand can be the complement of a coding strand or the complement of a non-coding strand. When referring to double stranded nucleic acids, the complement of a nucleic acid having SEQ ID NO: N refers to the complementary strand of the strand having SEQ ID NO: N or to any nucleic acid having the nucleotide sequence of the complementary strand of SEQ ID NO: N. When referring to a single stranded nucleic acid having the nucleotide sequence SEQ ID NO: N, the complement of this nucleic acid is a nucleic acid having a nucleotide sequence which is complementary to that of SEQ ID NO: N. The nucleotide sequences and complementary sequences thereof are always given in the 5′ to 3′ direction. The term “complement” and “reverse complement” are used interchangeably herein.

A “non-human animal” of the invention can include mammals such as rodents, non-human primates, sheep, goats, horses, dogs, cows, chickens, amphibians, reptiles, etc. Preferred non-human animals are selected from the rodent family including rat and mouse, most preferably mouse, though transgenic amphibians, such as members of the Xenopus genus, and transgenic chickens can also provide important tools for understanding and identifying agents which can affect, for example, embryogenesis and tissue formation. The term “chimeric animal” is used herein to refer to animals in which an exogenous sequence is found, or in which an exogenous sequence is expressed in some but not all cells of the animal. The term “tissue-specific chimeric animal” indicates that an exogenous sequence is present and/or expressed or disrupted in some tissues, but not others.

The term “oligonucleotide” is intended to include and single- or double stranded DNA or RNA. Oligonucleotides can be naturally occurring or synthetic, but are typically prepared by synthetic means. Preferred oligonucleotides of the invention include segments of a gene listed in Tables 1-5 or their complements, which include and/or flank any one of the polymorphic sites shown in Tables 1-5. The segments can be between 5 and 250 bases, and, in specific embodiments, are between 5-10, 5-20, 10-20, 10-50, 20-50 or 10-100 bases. For example, the segments can be 21 bases. The polymorphic site can occur within any position of the segment or a region next to the segment. The segments can be from any of the allelic forms of the gene sequences shown in Tables 1-5.

The term “operably-linked” is intended to mean that the 5′ upstream regulatory element is associated with a nucleic acid in such a manner as to facilitate transcription of the nucleic acid from the 5′ upstream regulatory element.

The term “polymorphism” refers to the coexistence of more than one form of a gene or portion thereof. A portion of a gene of which there are at least two different forms, i.e., two different nucleotide sequences, is referred to as a “polymorphic region of a gene.” A polymorphic locus can be a single nucleotide, the identity of which differs in the other alleles. A polymorphic locus can also be more than one nucleotide long. The allelic form occurring most frequently in a selected population is often referred to as the reference and/or wildtype form. Other allelic forms are typically designated or alternative or variant alleles. Diploid organisms may be homozygous or heterozygous for allelic forms. A diallelic or biallelic polymorphism has two forms. A trialleleic polymorphism has three forms.

A “polymorphic gene” refers to a gene having at least one polymorphic region.

The term “primer” as used herein, refers to a single-stranded oligonucleotide which acts as a point of initiation of template-directed DNA synthesis under appropriate conditions (e.g., in the presence of four different nucleoside triphosphates and as agent for polymerization, such as DNA or RNA polymerase or reverse transcriptase) in an appropriate buffer and at a suitable temperature. The length of a primer may vary but typically ranges from 15 to 30 nucleotides. A primer need not match the exact sequence of a template, but must be sufficiently complementary to hybridize with the template.

The term “primer pair” refers to a set of primers including an upstream primer that hybridizes with the 3′ end of the complement of the DNA sequence to be amplified and a downstream primer that hybridizes with the 3′ end of the sequence to be amplified.

The terms “protein”, “polypeptide” and “peptide” are used interchangeably herein when referring to a gene product.

The term “recombinant protein” refers to a polypeptide which is produced by recombinant DNA techniques, wherein generally, DNA encoding the polypeptide is inserted into a suitable expression vector which is in turn used to transform a host cell to produce the heterologous protein.

A “regulatory element”, also termed herein “regulatory sequence” is intended to include elements which are capable of modulating transcription from a 5′ upstream regulatory sequence, including, but not limited to a basic promoter, and include elements such as enhancers and silencers. The term “enhancer”, also referred to herein as “enhancer element”, is intended to include regulatory elements capable of increasing, stimulating, or enhancing transcription from a 5′ upstream regulatory element, including a basic promoter. The term “silencer”, also referred to herein as “silencer element” is intended to include regulatory elements capable of decreasing, inhibiting, or repressing transcription from a 5′ upstream regulatory element, including a basic promoter. Regulatory elements are typically present in 5′ flanking regions of genes. Regulatory elements also may be present in other regions of a gene, such as introns. Thus, it is possible that a gene has regulatory elements located in introns, exons, coding regions, and 3′ flanking sequences. Such regulatory elements are also intended to be encompassed by the present invention and can be identified by any of the assays that can be used to identify regulatory elements in 5′ flanking regions of genes.

The term “regulatory element” further encompasses “tissue specific” regulatory elements, i.e., regulatory elements which effect expression of an operably linked DNA sequence preferentially in specific cells (e.g., cells of a specific tissue). gene expression occurs preferentially in a specific cell if expression in this cell type is significantly higher than expression in other cell types. The term “regulatory element” also encompasses non-tissue specific regulatory elements, i.e., regulatory elements which are active in most cell types. Furthermore, a regulatory element can be a constitutive regulatory element, i.e., a regulatory element which constitutively regulates transcription, as opposed to a regulatory element which is inducible, i.e., a regulatory element which is active primarily in response to a stimulus. A stimulus can be, e.g., a molecule, such as a protein, hormone, cytokine, heavy metal, phorbol ester, cyclic AMP (cAMP), or retinoic acid.

Regulatory elements are typically bound by proteins, e.g., transcription factors. The term “transcription factor” is intended to include proteins or modified forms thereof, which interact preferentially with specific nucleic acid sequences, i.e., regulatory elements, and which in appropriate conditions stimulate or repress transcription. Some transcription factors are active when they are in the form of a monomer. Alternatively, other transcription factors are active in the form of a dimer consisting of two identical proteins or different proteins (heterodimer). Modified forms of transcription factors are intended to refer to transcription factors having a postranslational modification, such as the attachment of a phosphate group. The activity of a transcription factor is frequently modulated by a postranslational modification. For example, certain transcription factors are active only if they are phosphorylated on specific residues. Alternatively, transcription factors can be active in the absence of phosphorylated residues and become inactivated by phosphorylation. A list of known transcription factors and their DNA binding site can be found, e.g., in public databases, e.g., TFMATRIX Transcription Factor Binding Site Profile database.

The term “single nucleotide polymorphism” (SNP) refers to a polymorphic site occupied by a single nucleotide, which is the site of variation between allelic sequences. The site is usually preceded by and followed by highly conserved sequences of the allele (e.g., sequences that vary in less than 1/100 or 1/1000 members of a population). A SNP usually arises due to substitution of one nucleotide for another at the polymorphic site. SNPs can also arise from a deletion of a nucleotide or an insertion of a nucleotide relative to a reference allele. Typically the polymorphic site is occupied by a base other than the reference base. For example, where the reference allele contains the base “T” (thymidine) at the polymorphic site, the altered allele can contain a “C” (cytidine), “G” (guanine), or “A” (adenine) at the polymorphic site.

SNP's may occur in protein-coding nucleic acid sequences, in which case they may give rise to a defective or otherwise variant protein, or genetic disease. Such a SNP may alter the coding sequence of the gene and therefore specify another amino acid (a “missense” SNP) or a SNP may introduce a stop codon (a “nonsense” SNP). When a SNP does not alter the amino acid sequence of a protein, the SNP is called “silent.” SNP's may also occur in noncoding regions of the nucleotide sequence. This may result in defective protein expression, e.g., as a result of alternative spicing, or it may have no effect.

As used herein, the term “specifically hybridizes” or “specifically detects” refers to the ability of a nucleic acid molecule of the invention to hybridize to at least approximately 6, 12, 20, 30, 40, 50, 60, 70, 80, 90, 100, 110, 120, 130 or 140 consecutive nucleotides of either strand of a gene listed in Tables 1-5.

As used herein, the term “transfection” means the introduction of a nucleic acid, e.g., an expression vector, into a recipient cell by nucleic acid-mediated gene transfer. The term “transduction” is generally used herein when the transfection with a nucleic acid is by viral delivery of the nucleic acid. “Transformation”, as used herein, refers to a process in which a cell's genotype is changed as a result of the cellular uptake of exogenous DNA or RNA, and, for example, the transformed cell expresses a recombinant form of a polypeptide or, in the case of anti-sense expression from the transferred gene, the expression of a naturally-occurring form of the recombinant protein is disrupted.

As used herein, the term “transgene” refers to a nucleic acid sequence which has been genetic-engineered into a cell. Daughter cells deriving from a cell in which a transgene has been introduced are also said to contain the transgene (unless it has been deleted). A transgene can encode, e.g., a polypeptide, or an antisense transcript, partly or entirely heterologous, i.e., foreign, to the transgenic animal or cell into which it is introduced, or, is homologous to an endogenous gene of the transgenic animal or cell into which it is introduced, but which is designed to be inserted, or is inserted, into the animal's genome in such a way as to alter the genome of the cell into which it is inserted (e.g., it is inserted at a location which differs from that of the natural gene or its insertion results in a knockout). Alternatively, a transgene can also be present in an episome. A transgene can include one or more transcriptional regulatory sequence and any other nucleic acid, (e.g. intron), that may be necessary for optimal expression of a selected nucleic acid.

A “transgenic animal” refers to any animal, preferably a non-human animal, e.g. a mammal, bird or an amphibian, in which one or more of the cells of the animal contain heterologous nucleic acid introduced by genetic engineering, such as by transgenic techniques well known in the art. The nucleic acid is introduced into the cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extrachromosomally replicating DNA. In the typical transgenic animals described herein, the transgene causes cells to express a recombinant form of one of a protein, e.g. either agonistic or antagonistic forms. However, transgenic animals in which the recombinant gene is silent are also contemplated, as for example, the FLP or CRE recombinase dependent constructs described below. Moreover, “transgenic animal” also includes those recombinant animals in which gene disruption of one or more genes is caused by human intervention, including both recombination and antisense techniques.

The term “treatment,” or “treating” as used herein, is defined as the application or administration of a therapeutic agent to a subject, implementation of lifestyle changes (e.g., changes in diet or environment), administration of medication, e.g. lipid modulating medication, use of medical devices, such as, but not limited to, stents, defibrillators, and angioplasty devices, or any combination thereof or surgical procedures such as percutaneous transluminal coronary balloon angioplasty (PTCA) or laser angioplasty, defibrillators, implantation of a stent, or other surgical intervention, such as, for example, coronary bypass grafting (CABG), or any combination thereof, or application or administration of a therapeutic agent to an isolated tissue or cell line from a subject, who has a disease or disorder, a symptom of disease or disorder or a predisposition toward a disease or disorder, with the purpose to cure, heal, alleviate, relieve, alter, remedy, ameliorate, improve or affect the disease or disorder, the symptoms of the disease or disorder, or the predisposition toward disease. The medical devices described in the methods of the invention can also be used in combination with a modulator of gene expression or polypeptide activity. “Modulators of gene expression,” as used herein include, for example, nucleic acid molecules, antisense nucleic acid molecules, ribozymes, or a small molecules. “Modulators of polypeptide activity” include, for example, antibodies or proteins or polypeptides.

As used herein, the term “vector” refers to a nucleic acid molecule capable of transporting or replicating another nucleic acid to which it has been linked. One type of preferred vector is an episome, i.e., a nucleic acid capable of extra-chromosomal replication. Preferred vectors are those capable of autonomous replication and/or expression of nucleic acids to which they are linked. Vectors capable of directing the expression of genes to which they are operatively-linked are referred to herein as “expression vectors”. In general, expression vectors of utility in recombinant DNA techniques are often in the form of “plasmids” which refer generally to circular double stranded DNA circles which, in their vector form are not physically linked to the host chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

Polymorphisms of the Invention

The nucleic acid molecules of the present invention include specific allelic variants of the genes in Tables 1-5, which differ from the respective reference sequence, or at least a portion thereof, having a polymorphic region. The preferred nucleic acid molecules of the present invention comprise sequences having the polymorphisms shown in Table 5, and those in linkage disequilibrium therewith. The invention further comprises isolated nucleic acid molecules complementary to nucleic acid molecules comprising the polymorphisms of the present invention. Nucleic acid molecules of the present invention can function as probes or primers, e.g., in methods for determining the allelic identity of a polymorphic region of the genes identified in Tables 1-5. The nucleic acids of the invention can also be used, either in combination with each other or in combination with other SNPs in these genes or other genes, to determine whether a subject is or is not at risk of developing a disease associated with a specific allelic variant of a polymorphic region of a gene identified in Tables 1-5, e.g., an abnormal lipid level. The nucleic acids of the invention can further be used to prepare or express polypeptides encoded by specific alleles of the genes identified in Tables 1-5, such as mutant alleles. Such nucleic acids can be used in gene therapy. Polypeptides encoded by specific alleles, such as mutant polypeptides, can also be used in therapy or for preparing reagents, e.g., antibodies, for detecting proteins encoded by these alleles. Accordingly, such reagents can be used to detect mutant proteins encoded by the genes identified in Tables 1-5.

As described herein, allelic variants of the human genes listed in Tables 1-5 which are associated with abnormal lipid levels have been identified. The invention is intended to encompass the allelic variants as well as those in linkage disequilibrium which can be identified, e.g., according to the methods described herein (see, for example, the SNPs listed in Table 4).

The invention also provides isolated nucleic acids comprising at least one polymorphic region of a gene listed in Tables 1 having a nucleotide sequence which differs from the reference nucleotide sequence. Preferred nucleic acids can have a polymorphic region in an upstream regulatory element, an exon, an intron, or in the 3′ UTR.

The nucleic acid molecules of the invention can be single stranded DNA (e.g., an oligonucleotide), double stranded DNA (e.g., double stranded oligonucleotide) or RNA. Preferred nucleic acid molecules of the invention can be used as probes or primers. Primers of the invention refer to nucleic acids which hybridize to a nucleic acid sequence which is adjacent to the region of interest or which covers the region of interest and is extended. As used herein, the term “hybridizes” is intended to describe conditions for hybridization and washing under which nucleotide sequences that are significantly identical or homologous to each other remain hybridized to each other. Preferably, the conditions are such that sequences at least about 70%, more preferably at least about 80%, even more preferably at least about 85% or 90% identical to each other remain hybridized to each other. Such stringent conditions vary according to the length of the involved nucleotide sequence but are known to those skilled in the art and can be found or determined based on teachings in Current Protocols in Molecular Biology, Ausubel et al., eds., John Wiley & Sons, Inc. (1995), sections 2, 4 and 6. Additional stringent conditions and formulas for determining such conditions can be found in Molecular Cloning: A Laboratory Manual, Sambrook et al., Cold Spring Harbor Press, Cold Spring Harbor, N.Y. (1989), chapters 7, 9 and 11. A preferred, non-limiting example of stringent hybridization conditions for hybrids that are at least basepairs in length includes hybridization in 4× sodium chloride/sodium citrate (SSC), at about 65-70° C. (or hybridization in 4×SSC plus 50% formamide at about 42-50° C.) followed by one or more washes in 1×SSC, at about 65-70° C. A preferred, non-limiting example of highly stringent hybridization conditions for such hybrids includes hybridization in 1×SSC, at about 65-70° C. (or hybridization in 1×SSC plus 50% formamide at about 42-50° C.) followed by one or more washes in 0.3×SSC, at about 65-70° C. A preferred, non-limiting example of reduced stringency hybridization conditions for such hybrids includes hybridization in 4×SSC, at about 50-60° C. (or alternatively hybridization in 6×SSC plus 50% formamide at about 40-45° C.) followed by one or more washes in 2×SSC, at about 50-60° C. Ranges intermediate to the above-recited values, e.g., at 65-70° C. or at 42-50° C. are also intended to be encompassed by the present invention. SSPE (1×SSPE is 0.15M NaCl, 10 mM NaH₂PO₄, and 1.25 mM EDTA, pH 7.4) can be substituted for SSC (1×SSC is 0.15M NaCl and 15 mM sodium citrate) in the hybridization and wash buffers; washes are performed for 15 minutes each after hybridization is complete.

The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.)=2(# of A+T bases)+4(# of G+C bases). For hybrids between 18 and 49 base pairs in length, Tₘ(° C.)=81.5+16.6(log₁₀[Na⁺])+0.41(% G+C)−(600/N), where N is the number of bases in the hybrid, and [Na⁺] is the concentration of sodium ions in the hybridization buffer ([Na⁺] for 1×SSC=0.165 M). It will also be recognized by the skilled practitioner that additional reagents may be added to hybridization and/or wash buffers to decrease non-specific hybridization of nucleic acid molecules to membranes, for example, nitrocellulose or nylon membranes, including but not limited to blocking agents (e.g., BSA or salmon or herring sperm carrier DNA), detergents (e.g., SDS), chelating agents (e.g., EDTA), Ficoll, PVP and the like. When using nylon membranes, in particular, an additional preferred, non-limiting example of stringent hybridization conditions is hybridization in 0.25-0.5M NaH₂PO₄, 7% SDS at about 65° C., followed by one or more washes at 0.02M NaH₂PO₄, 1% SDS at 65° C., see e.g., Church and Gilbert (1984) Proc. Natl. Acad. Sci. USA 81:1991-1995, (or alternatively 0.2×SSC, 1% SDS).

A primer or probe can be used alone in a detection method, or a primer can be used together with at least one other primer or probe in a detection method. Primers can also be used to amplify at least a portion of a nucleic acid. Probes of the invention refer to nucleic acids which hybridize to the region of interest and which are not further extended. For example, a probe is a nucleic acid which specifically hybridizes to a polymorphic region of a gene listed in Tables 1-5, and which by hybridization or absence of hybridization to the DNA of a subject or the type of hybrid formed will be indicative of the identity of the allelic variant of the polymorphic region of the gene.

Numerous procedures for determining the nucleotide sequence of a nucleic acid molecule, or for determining the presence of mutations in nucleic acid molecules include a nucleic acid amplification step, which can be carried out by, e.g., polymerase chain reaction (PCR). Accordingly, in one embodiment, the invention provides primers for amplifying portions of a gene listed in Tables 1-5, such as portions of exons and/or,portions of introns. In a preferred embodiment, the exons and/or sequences adjacent to the exons of a human gene listed in Tables 1-5 will be amplified to, e.g., detect which allelic variant, if any, of a polymorphic region is present in the gene of a subject. Preferred primers comprise a nucleotide sequence complementary to a specific allelic variant of a polymorphic region of a gene listed in Tables 1-5 and of sufficient length to selectively hybridize with a gene listed in Tables 1-5, or a combination thereof. In a preferred embodiment, the primer, e.g., a substantially purified oligonucleotide, comprises a region having a nucleotide sequence which hybridizes under stringent conditions to about 6, 8, 10, or 12, preferably 25, 30, 40, 50, or 75 consecutive nucleotides of a gene listed in Tables 1-5. In an even more preferred embodiment, the primer is capable of hybridizing to a nucleotide sequence of a gene listed in Tables 1-5, complements thereof, allelic variants thereof, or complements of allelic variants thereof. For example, primers comprising a nucleotide sequence of at least about 15 consecutive nucleotides, at least about 25 nucleotides or having from about 15 to about 20 nucleotides set forth in SEQ ID NOs: 1-26, or the complement thereof are provided by the invention. Primers having a sequence of more than about 25 nucleotides are also within the scope of the invention. Preferred primers of the invention are primers that can be used in PCR for amplifying each of the exons of a gene listed in Tables 1-5.

Primers can be complementary to nucleotide sequences located close to each other or further apart, depending on the use of the amplified DNA. For example, primers can be chosen such that they amplify DNA fragments of at least about 10 nucleotides or as much as several kilobases. Preferably, the primers of the invention will hybridize selectively to nucleotide sequences located about 150 to about 350 nucleotides apart.

For amplifying at least a portion of a nucleic acid, a forward primer (i.e., 5′ primer) and a reverse primer (i.e., 3′ primer) will preferably be used. Forward and reverse primers hybridize to complementary strands of a double stranded nucleic acid, such that upon extension from each primer, a double stranded nucleic acid is amplified. A forward primer can be a primer having a nucleotide sequence or a portion of a nucleotide sequence shown in Tables 1-5 (SEQ ID NOs: 1-26). A reverse primer can be a primer having a nucleotide sequence or a portion of the nucleotide sequence that is complementary to a nucleotide sequence shown in Tables 1-5 (SEQ ID NOs: 1-26).

Yet other preferred primers of the invention are nucleic acids which are capable of selectively hybridizing to an allelic variant of a polymorphic region of a gene listed in Tables 1-5. Thus, such primers can be specific for the sequence of a gene listed in Tables 1-5, so long as they have a nucleotide sequence which is capable of hybridizing to a gene listed in Tables 1-5. Preferred primers are capable of specifically hybridizing to the allelic variants listed in Tables 1-5 (SEQ ID NOs: 1-26). Such primers can be used, e.g., in sequence specific oligonucleotide priming as described further herein.

Other preferred primers used in the methods of the invention are nucleic acids which are capable of hybridizing to the reference sequence of a gene listed in Tables 1-5, thereby detecting the presence of the reference allele of an allelic variant or the absence of a variant allele of an allelic variant in a gene listed in Tables 1-5. Such primers can be used in combination, e.g., primers specific for the variant polynucleotide of a gene listed in Tables 1-5 can be used in combination. The sequences of primers specific for the reference sequences comprising a gene listed in Tables 1-5 will be readily apparent to one of skill in the art.

The nucleic acids of the invention can also be used as probes, e.g., in therapeutic and diagnostic assays. For instance, the present invention provides a probe comprising a substantially purified oligonucleotide, which oligonucleotide comprises a region having a nucleotide sequence that is capable of hybridizing specifically to a region of a gene listed in Tables 1-5 which is polymorphic (SEQ ID NOs: 1-26). In an even more preferred embodiment of the invention, the probes are capable of hybridizing specifically to one allelic variant of a gene listed in Tables 1-5 having a nucleotide sequence which differs from the nucleotide sequence set forth in the corresponding reference sequence. Such probes can then be used to specifically detect which allelic variant of a polymorphic region of a gene listed in Tables 1-5 is present in a subject. The polymorphic region can be located in the 3′ UTR, 5′ upstream regulatory element, exon, or intron sequences of a gene listed in Tables 1-5.

Particularly, preferred probes of the invention have a number of nucleotides sufficient to allow specific hybridization to the target nucleotide sequence. Where the target nucleotide sequence is present in a large fragment of DNA, such as a genomic DNA fragment of several tens or hundreds of kilobases, the size of the probe may have to be longer to provide sufficiently specific hybridization, as compared to a probe which is used to detect a target sequence which is present in a shorter fragment of DNA. For example, in some diagnostic methods, a portion of a gene listed in Tables 1-5 may first be amplified and thus isolated from the rest of the chromosomal DNA and then hybridized to a probe. In such a situation, a shorter probe will likely provide sufficient specificity of hybridization. For example, a probe having a nucleotide sequence of about 10 nucleotides may be sufficient.

In preferred embodiments, the probe or primer further comprises a label attached thereto, which, e.g., is capable of being detected, e.g. the label group is selected from amongst radioisotopes, fluorescent compounds, enzymes, and enzyme co-factors.

In a preferred embodiment of the invention, the isolated nucleic acid, which is used, e.g., as a probe or a primer, is modified, so as to be more stable than naturally occurring nucleotides. Exemplary nucleic acid molecules which are modified include phosphoramidate, phosphothioate and methylphosphonate analogs of DNA (see also U.S. Pat. Numbers 5,176,996; 5,264,564; and 5,256,775).

The nucleic acids of the invention can also be modified at the base moiety, sugar moiety, or phosphate backbone, for example, to improve stability of the molecule. The nucleic acids, e.g., probes or primers, may include other appended groups such as peptides (e.g., for targeting host cell receptors in vivo), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. WO88/09810, published Dec. 15, 1988), hybridization-triggered cleavage agents. (See, e.g., Krol et al., 1988, BioTechniques 6:958-976) or intercalating agents (see, e.g., Zon, 1988, Pharm. Res. 5:539-549). To this end, the nucleic acid of the invention may be conjugated to another molecule, e.g., a peptide, hybridization triggered cross-linking agent, transport agent, hybridization-triggered cleavage agent, etc.

The isolated nucleic acid comprising an intronic sequence of a gene listed in Tables 1-5 may comprise at least one modified base moiety which is selected from the group including but not limited to 5-fluorouracil, 5-bromouracil, 5-chlorouracil, 5-iodouracil, hypoxanthine, xantine, 4-acetylcytidine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytidine, 5-methylcytidine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytidine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The isolated nucleic acid may also comprise at least one modified sugar moiety selected from the group including but not limited to arabinose, 2-fluoroarabinose, xylulose, and hexose.

In yet another embodiment, the nucleic acid comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet a further embodiment, the nucleic acid is an α-anomeric oligonucleotide. An α-anomeric oligonucleotide forms specific double-stranded hybrids with complementary RNA in which, contrary to the usual β-units, the strands run parallel to each other (Gautier et al., 1987, Nucl. Acids Res. 15:6625-6641). The oligonucleotide is a 2′-0-methylribonucleotide (Inoue et al., 1987, Nucl. Acids Res. 15:6131-6148), or a chimeric RNA-DNA analogue (Inoue et al., 1987, FEBS Lett. 215:327-330).

Any nucleic acid fragment of the invention can be prepared according to methods well known in the art and described, e.g., in Sambrook, J. Fritsch, E. F., and Maniatis, T. (1989) Molecular Cloning: A Laboratory Manual, Cold Spring Harbor Laboratory Press, Cold Spring Harbor, N.Y. For example, discrete fragments of the DNA can be prepared and cloned using restriction enzymes. Alternatively, discrete fragments can be prepared using the Polymerase Chain Reaction (PCR) using primers having an appropriate sequence.

Oligonucleotides of the invention may be synthesized by standard methods known in the art, e.g. by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16:3209), methylphosphonate oligonucleotides can be prepared by use of controlled pore glass polymer supports (Sarin et al., 1988, Proc. Natl. Acad Sci. U.S.A. 85:7448-7451), etc.

The invention also provides vectors and plasmids comprising the nucleic acids of the invention. For example, in one embodiment, the invention provides a vector comprising at least a portion of a gene listed in Tables 1-5 comprising a polymorphic region. Thus, the invention provides vectors for expressing at least a portion of the newly identified allelic variants of the human genes listed in Tables 1-5, as well as other allelic variants, comprising a nucleotide sequence which is different from the nucleotide sequences disclosed in the reference sequences. The allelic variants can be expressed in eukaryotic cells, e.g., cells of a subject, e.g., a mammalian subject, or in prokaryotic cells.

In one embodiment, the vector comprising at least a portion of an allele is introduced into a host cell, such that a protein encoded by the allele is synthesized. The protein produced can be used, e.g., for the production of antibodies, which can be used, e.g., in methods for detecting mutant forms of proteins encoded by the genes listed in Tables 1-5. Alternatively, the vector can be used for gene therapy, and be, e.g., introduced into a subject to produce protein. Host cells comprising a vector having at least a portion of a gene listed in Tables 1-5 are also within the scope of the invention.

Polypeptides of the invention

The present invention provides isolated polypeptides encoded by the genes listed in Tables 1-5, such as polypeptides which are encoded by specific allelic variants of these genes.

In one embodiment, the polypeptides encoded by the genes listed in Tables 1-5 are isolated from, or otherwise substantially free of other cellular proteins. The term “substantially free of other cellular proteins” (also referred to herein as “contaminating proteins”) or “substantially pure or purified preparations” are defined as encompassing preparations of polypeptides encoded by the genes listed in Tables 1-5 having less than about 20% (by dry weight) contaminating protein, and preferably having less than about 5% contaminating protein. It will be appreciated that functional forms of the subject polypeptides can be prepared, for the first time, as purified preparations by using a cloned gene as described herein.

Preferred proteins of the invention have an amino acid sequence which is at least about 60%, 70%, 80%, 85%, 90%, or 95% identical or homologous to the amino acid sequence of the proteins encoded by the genes listed in Tables 1-5. Even more preferred proteins comprise an amino acid sequence which is at least about 95%, 96%, 97%, 98%, or 99% homologous or identical to polypeptides encoded by the genes listed in Tables 1-5. Such proteins can be recombinant proteins, and can be, e.g., produced in vitro from nucleic acids comprising a specific allele of a polymorphic region. For example, recombinant polypeptides preferred by the present invention can be encoded by a nucleic acid which comprises a sequence which is at least 85% homologous and more preferably 90% homologous and most preferably 95% homologous with a reference nucleotide sequence of a gene listed in Tables 1-5, as set forth herein, and comprises an allele of a polymorphic region that differs from that set forth in Tables 1-5. Polypeptides which are encoded by a nucleic acid comprising a sequence that is at least about 98-99% homologous with a reference nucleotide sequence of a gene listed in Tables 1-5 and comprises an allele of a polymorphic region that differs from that set forth in a reference nucleotide sequence of a gene listed in Tables 1-5 are also within the scope of the invention.

In a preferred embodiment, a protein of the present invention is a mammalian protein. In an even more preferred embodiment, the protein is a human protein.

The invention also provides peptides that preferably are capable of functioning in one of either role of an agonist or antagonist of at least one biological activity of a wild-type (“normal”) protein encoded by a gene listed in Tables 1-5. The term “evolutionarily related to,” with respect to amino acid sequences of proteins encoded by the genes listed in Tables 1-5, refers to both polypeptides having amino acid sequences found in human populations, and also to artificially produced mutational variants of human polypeptides encoded by the genes listed in Tables 1-5 which are derived, for example, by combinatorial mutagenesis.

Full length proteins or fragments corresponding to one or more particular motifs and/or domains or to arbitrary sizes, for example, at least 5, 10, 25, 50, 75 and 100, amino acids in length of proteins encoded by the genes listed in Tables 1-5 are within the scope of the present invention.

Isolated peptides or polypeptides encoded by the genes listed in Tables 1-5 can be obtained by screening peptides recombinantly produced from the corresponding fragment of the nucleic acid encoding such peptides. In addition, such peptides and polypeptides can be chemically synthesized using techniques known in the art such as conventional Merrifield solid phase f-Moc or t-Boc chemistry. For example, an peptide or polypeptide of the present invention may be arbitrarily divided into fragments of desired length with no overlap of the fragments, or preferably divided into overlapping fragments of a desired length. The fragments can be produced (recombinantly or by chemical synthesis) and tested to identify those peptides or polypeptides which can function as either agonists or antagonists of a wild-type (e.g., “normal”) protein encoded by a gene listed in Tables 1-5.

In general, peptides and polypeptides referred to herein as having an activity (e.g., are “bioactive”) of a protein encoded by a gene listed in Tables 1-5 are defined as peptides and polypeptides which mimic or antagonize all or a portion of the biological/biochemical activities of a protein encoded by a gene listed in Tables 1-5, such as the ability to bind ligands. Other biological activities of the subject proteins are described herein or will be reasonably apparent to those skilled in the art. According to the present invention, a peptide or polypeptide has biological activity if it is a specific agonist or antagonist of a naturally-occurring form of a protein encoded by a gene listed in Tables 1-5.

Assays for determining whether a protein encoded by a gene listed in Tables 1-5 or variant thereof, has one or more biological activities are well known in the art.

Other preferred proteins of the invention are those encoded by the nucleic acids set forth in the section pertaining to nucleic acids of the invention. In particular, the invention provides fusion proteins, e.g., immunoglobulin fusion proteins comprising a protein encoded by a gene listed in Tables 1-5. Such fusion proteins can provide, e.g., enhanced stability and solubility of a protein encoded by a gene listed in Tables 1-5 and may thus be useful in therapy. Fusion proteins can also be used to produce an immunogenic fragment of a protein encoded by a gene listed in Tables 1-5. For example, the VP6 capsid protein of rotavirus can be used as an immunologic carrier protein for portions of a polypeptide, either in the monomeric form or in the form of a viral particle. The nucleic acid sequences corresponding to the portion of a subject protein to which antibodies are to be raised can be incorporated into a fusion gene construct which includes coding sequences for a late vaccinia virus structural protein to produce a set of recombinant viruses expressing fusion proteins comprising epitopes of a protein encoded by a gene listed in Tables 1-5 as part of the virion. It has been demonstrated with the use of immunogenic fusion proteins utilizing the Hepatitis B surface antigen fusion proteins that recombinant Hepatitis B virions can be utilized in this role as well. Similarly, chimeric constructs coding for fusion proteins containing a portion of a protein encoded by a gene listed in Tables 1-5 and the poliovirus capsid protein can be created to enhance immunogenicity of the set of polypeptide antigens (see, for example, EP Publication No: 0259149; and Evans et al. (1989) Nature 339:385; Huang et al. (1988) J. Virol. 62:3855; and Schlienger et al. (1992) J. Virol. 66:2).

The Multiple antigen peptide system for peptide-based immunization can also be utilized to generate an immunogen, wherein a desired portion of a polypeptide encoded by a gene listed in Tables 1-5 is obtained directly from organo-chemical synthesis of the peptide onto an oligomeric branching lysine core (see, for example, Posnett et al. (1988) JBC 263:1719 and Nardelli et al. (1992) J. Immunol. 148:914). Antigenic determinants of proteins encoded by genes listed in Tables 1-5 can also be expressed and presented by bacterial cells.

Fusion proteins can also facilitate the expression of proteins including the polypeptides encoded by a gene listed in Tables 1-5. For example, polypeptides can be generated as glutathione-S-transferase (GST-fusion) proteins. Such GST-fusion proteins can be easily purified, as for example by the use of glutathione-derivatized matrices (see, for example, Current Protocols in Molecular Biology, eds. Ausubel et al. (N.Y.: John Wiley & Sons, 1991)) and used subsequently to yield purified polypeptides encoded by a gene listed in Tables 1-5.

The present invention further pertains to methods of producing the subject polypeptides. For example, a host cell transfected with a nucleic acid vector directing expression of a nucleotide sequence encoding the subject polypeptides can be cultured under appropriate conditions to allow expression of the peptide to occur. Suitable media for cell culture are well known in the art. The recombinant polypeptide can be isolated from cell culture medium, host cells, or both using techniques known in the art for purifying proteins including ion-exchange chromatography, gel filtration chromatography, ultrafiltration, electrophoresis, and immunoaffinity purification with antibodies specific for such peptide. In a preferred embodiment, the recombinant polypeptide is a fusion protein containing a domain which facilitates its purification, such as GST fusion protein.

Moreover, it will be generally appreciated that, under certain circumstances, it may be advantageous to provide homologs of one of the subject polypeptides which function in a limited capacity as one of either an agonist (mimetic) or an antagonist, in order to promote or inhibit only a subset of the biological activities of the naturally-occurring form of the protein. Thus, specific biological effects can be elicited by treatment with a homolog of limited function, and with fewer side effects relative to treatment with agonists or antagonists which are directed to all of the biological activities of naturally occurring forms of proteins encoded by genes listed in Tables 1-5.

Homologs of each of the subject proteins can be generated by mutagenesis, such as by discrete point mutation(s), and/or by truncation. For instance, mutation can give rise to homologs which retain substantially the same, or merely a subset, of the biological activity of the polypeptide from which it was derived. Alternatively, antagonistic forms of the protein can be generated which are able to inhibit the function of the naturally occurring form of the protein, such as by competitively binding to a receptor of a protein encoded by a gene listed in Tables 1-5.

The recombinant polypeptides of the present invention also include homologs of polypeptides encoded by the genes listed in Tables 1-5 which differ from the reference protein, such as versions of the protein which are resistant to proteolytic cleavage, as for example, due to mutations which alter ubiquitination or other enzymatic targeting associated with the protein.

Polypeptides encoded by the genes listed in Tables 1-5 may also be chemically modified to create derivatives by forming covalent or aggregate conjugates with other chemical moieties, such as glycosyl groups, lipids, phosphate, acetyl groups and the like. Covalent derivatives of proteins encoded by the genes listed in Tables 1-5 can be prepared by linking the chemical moieties to functional groups on amino acid side-chains of the protein or at the N-terminus or at the C-terminus of the polypeptide.

Modification of the structure of the subject polypeptides can be for such purposes as enhancing therapeutic or prophylactic efficacy, stability (e.g., ex vivo shelf life and resistance to proteolytic degradation), or post-translational modifications (e.g., to alter phosphorylation pattern of protein). Such modified peptides, when designed to retain at least one activity of the naturally-occurring form of the protein, or to produce specific antagonists thereof, are considered functional equivalents of the polypeptides described in more detail herein. Such modified peptides can be produced, for instance, by amino acid substitution, deletion, or addition. The substitutional variant may be a substituted conserved amino acid or a substituted non-conserved amino acid.

For example, it is reasonable to expect that an isolated replacement of a leucine with an isoleucine or valine, an aspartate with a glutamate, a threonine with a serine, or a similar replacement of an amino acid with a structurally related amino acid (i.e., isosteric and/or isoelectric mutations) will not have a major effect on the biological activity of the resulting molecule. Conservative replacements are those that take place within a family of amino acids that are related in their side chains. Genetically encoded amino acids can be divided into four families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. In similar fashion, the amino acid repertoire can be grouped as (1) acidic=aspartate, glutamate; (2) basic=lysine, arginine histidine, (3) aliphatic=glycine, alanine, valine, leucine, isoleucine, serine, threonine, with serine and threonine optionally be grouped separately as aliphatic-hydroxyl; (4) aromatic=phenylalanine, tyrosine, tryptophan; (5) amide=asparagine, glutamine; and (6) sulfur-containing=cysteine and methionine. (see, for example, Biochemistry, 2^{nd} ed., Ed. by L. Stryer, WH Freeman and Co.: 1981). Whether a change in the amino acid sequence of a peptide results in a functional homolog (e.g., functional in the sense that the resulting polypeptide mimics or antagonizes the wild-type form) can be readily determined by assessing the ability of the variant peptide to produce a response in cells in a fashion similar to the wild-type protein, or competitively inhibit such a response. Polypeptides in which more than one replacement has taken place can readily be tested in the same manner.

Methods

The invention further provides predictive medicine methods, which are based, at least in part, on the discovery of polymorphic regions which are associated with specific physiological states and/or diseases or disorders, e.g., abnormal lipid levels, e.g., abnormally low HDL-C level, or a disease or disorder associated with abnormal lipid levels, e.g., a vascular or metabolic disease or disorder. These methods can be used alone, or in combination with other predictive medicine methods, including the identification and analysis of known risk factors associated with vascular diseases or disorders, metabolic diseases or disorders, and/or abnormal lipid levels, e.g., phenotypic factors such as, for example, family history.

For example, information obtained using the diagnostic assays described herein (in combination with each other or in combination with information of another genetic defect which contributes to the same disease, e.g., abnormal lipid levels, e.g., abnormally low HDL-C level, or a disease or disorder associated with abnormal lipid levels, e.g., a vascular or metabolic disease or disorder), is useful for diagnosing or confirming that a subject has an allele of a polymorphic region (e.g., a specific allele listed in Table 5) which is associated with a particular disease or disorder, e.g., abnormal lipid levels, e.g., abnormally low HDL-C level, or a disease or disorder associated with abnormal lipid levels, e.g., a vascular or metabolic disease or disorder, or a combination of alleles which are associated with a particular disease or disorder. Moreover, the information obtained using the diagnostic assays described herein, in combination with each other or in combination with information of another genetic defect which contributes to the same disease, e.g., abnormal lipid levels, e.g., abnormally low HDL-C level, or a disease or disorder associated with abnormal lipid levels, e.g., a vascular or metabolic disease or disorder, can be used to predict whether or not a subject will benefit from further diagnostic evaluation for abnormal lipid levels, e.g., abnormally low HDL-C level, or a disease or disorder associated with abnormal lipid levels, e.g., a vascular or metabolic disease or disorder. Such further diagnostic evaluation includes, but is not limited to, cardiovascular imaging, such as angiography, cardiac ultrasound, coronary angiogram, magnetic resonance imagery, nuclear imaging, CT scan, myocardial perfusion imagery, or electrocardiogram, genetic analysis, e.g., identification of additional polymorphisms e.g., which contribute to the same disease(s), familial health history analysis, lifestyle analysis, or exercise stress tests, either alone or in combination. Furthermore, the diagnostic information obtained using the diagnostic assays described herein (in combination with each other or in combination with information of another genetic defect which contributes to the same disease, e.g., a vascular or metabolic disease or disorder, or low HDL-C), may be used to identify which subject will benefit from a particular clinical course of therapy useful for preventing, treating, ameliorating, or prolonging onset of the particular disease or disorder, e.g., abnormal lipid levels, e.g., abnormally low HDL-C level, or a vascular or metabolic disease or disorder in the particular subject. Clinical courses of therapy include, but are not limited to, administration of medication, e.g., lipid modulating medication, non-surgical intervention, surgical procedures such as percutaneous transluminal coronary angioplasty, laser angioplasty, implantation of a stent, coronary bypass grafting, implantation of a defibrillator, implantation of a pacemaker, and any combination thereof, and use of surgical and non-surgical medical devices used in the treatment of vascular disease, such as, for example, a defibrillator, a stent, a device used in coronary revascularization, a pacemaker, and any combination thereof. Medical devices may also be used in combination with a modulator of gene expression of the genes identified in Tables 1-5 or polypeptide activity of polypeptides encoded by the genes identified in Tables 1-5.

Alternatively, the information, alone or in combination with information of another genetic defect which contributes to the same disorder, e.g., abnormal lipid levels, e.g., abnormally low HDL-C level, or a disease or disorder associated with abnormal lipid levels, e.g., a vascular or metabolic disease or disorder, can be used prognostically for predicting whether a non-symptomatic subject is likely to develop a disease or condition which is associated with one or more specific alleles of polymorphic regions of a gene listed in Tables 1-5 in a subject. Based on the prognostic information, a health care provider can recommend a particular further diagnostic evaluation which will benefit the subject, or a particular clinical course of therapy, as described above. The information may also be used to predict the response of a female subject to HRT.

In addition, knowledge of the identity of one or more particular alleles in a subject (the genetic profile of a gene listed in Tables 1-5), allows customization of further diagnostic evaluation and/or a clinical course of therapy for a particular disease. For example, a subject's genetic profile or the genetic profile of a disease or disorder associated with a specific allele of a polymorphic region of a gene listed in Tables 1-5, e.g., abnormal lipid levels, e.g., abnormally low HDL-C level, or a disease or disorder associated with abnormal lipid levels, e.g., a vascular or metabolic disease or disorder, can enable a health care provider: 1) to more efficiently and cost-effectively identify means for further diagnostic evaluation, including, but not limited to, further genetic analysis, familial health history analysis, or use of imaging devices or procedures; 2) to more effectively prescribe a drug that will address the molecular basis of the disease or condition; 3) to more efficiently and cost-effectively identify an appropriate clinical course of therapy, including, but not limited to, lifestyle changes, medications, surgical or non-surgical medical devices, surgical or non-surgical intervention or procedures, or any combination thereof; and 4) to better determine the appropriate dosage of a particular drug or duration of a particular course of clinical therapy. For example, the expression level of proteins encoded by genes listed in Tables 1-5, alone or in conjunction with the expression level of other genes known to contribute to the same disease, can be measured in many subjects at various stages of the disease to generate a transcriptional or expression profile of the disease. Expression patterns of individual subjects can then be compared to the expression profile of the disease to determine the appropriate drug, dose to administer to the subject, or course of clinical therapy.

The ability to target populations expected to show the highest clinical benefit, based on the genetic profile, can enable: 1) the repositioning of marketed drugs, medical devices and surgical procedures for use in treating, preventing, or ameliorating abnormal lipid levels or vascular or metabolic diseases or disorders, or diagnostics, such as imaging devices or procedures, with disappointing market results; 2) the rescue of drug candidates whose clinical development has been discontinued as a result of safety or efficacy limitations, which are subject subgroup-specific; 3) an accelerated and less costly development for drug candidates and more optimal drug labeling (e.g., since the use of genes listed in Tables 1-5 as markers is useful for optimizing effective dose); and 4) an accelerated, less costly, and more effective selection of a particular course of clinical therapy suited to a particular subject.

These and other methods are described in further detail in the following sections.

A. Prognostic and Diagnostic Assays

The present methods provide means for determining if a subject has or is or is not at risk of developing a disease, condition or disorder that is associated a specific allele of a gene listed in Tables 1-5, or combinations thereof, e.g., abnormal lipid levels, e.g., abnormally low HDL-C level, or a disease or disorder associated with abnormal lipid levels, e.g., a vascular or metabolic disease or disorder. The present methods also provide means for determining if a subject, e.g., a female subject, e.g., a postmenopausal female subject, is at risk for developing abnormal lipid levels and/or diseases or disorders associated with abnormal lipid levels, e.g., vascular or metabolic disorders, in response to treatment with HRT.

The present invention provides methods for determining the molecular structure of a gene listed in Tables 1-5, e.g., a human gene, or a portion thereof. In one embodiment, determining the molecular structure of at least a portion of a gene listed in Tables 1-5 comprises determining the identity of the allelic variant of at least one polymorphic region of a gene listed in Tables 1-5, or the complement thereof. A polymorphic region of a gene listed in Tables 1-5 can be located in an exon, an intron, at an intron/exon border, or in the 5′ upstream regulatory element of the subject gene.

The invention provides methods for determining whether a subject has or is at risk of developing, a disease or disorder associated with a specific allelic variant of a polymorphic region of a gene listed in Tables 1-5. Such diseases can be associated with aberrant polypeptide activity, e.g., abnormal lipid levels, e.g., abnormally low HDL-C level, or a disease or disorder associated with abnormal lipid levels, e.g., a vascular or metabolic disease or disorder.

Analysis of one or more polymorphic regions in a subject can be useful for predicting whether a subject has or is likely to develop abnormal lipid levels, e.g., abnormally low HDL-C level, or a disease or disorder associated with abnormal lipid levels, e.g., a vascular or metabolic disease or disorder.

In preferred embodiments, the methods of the invention can be characterized as comprising detecting, in a sample of cells from the subject, the presence or absence of a specific allelic variant of one or more polymorphic regions of a gene listed in Tables 1-5. The allelic differences can be: (i) a difference in the identity of at least one nucleotide or (ii) a difference in the number of nucleotides, which difference can be a single nucleotide or several nucleotides. The invention also provides methods for detecting differences in a gene listed in Tables 1-5 such as chromosomal rearrangements, e.g., chromosomal dislocation. The invention can also be used in prenatal diagnostics.

A preferred detection method is allele specific hybridization using probes overlapping the polymorphic site and having about 5, 10, 20, 25, or 30 nucleotides around the polymorphic region. In a preferred embodiment of the invention, several probes capable of hybridizing specifically to allelic variants are attached to a solid phase support, e.g., a “chip”. Oligonucleotides can be bound to a solid support by a variety of processes, including lithography. For example a chip can hold up to 250,000 oligonucleotides (GeneChip, Affymetrix). Mutation detection analysis using these chips comprising oligonucleotides, also termed “DNA probe arrays” is described e.g. in Cronin et al. (1996) Human Mutation 7:244. In one embodiment, a chip comprises all the allelic variants of at least one polymorphic region of a gene. The solid phase support is then contacted with a test nucleic acid and hybridization to the specific probes is detected. Accordingly, the identity of numerous allelic variants of one or more genes can be identified in a simple hybridization experiment. For example, the identity of the allelic variant of the nucleotide polymorphism in the 5′ upstream regulatory element can be determined in a single hybridization experiment.

In other detection methods, it is necessary to first amplify at least a portion of a gene prior to identifying the allelic variant. Amplification can be performed, e.g., by PCR and/or LCR (see Wu and Wallace, (1989) Genomics 4:560), according to methods known in the art. In one embodiment, genomic DNA of a cell is exposed to two PCR primers and amplification for a number of cycles sufficient to produce the required amount of amplified DNA. In preferred embodiments, the primers are located between 150 and 350 base pairs apart.

Alternative amplification methods include: self sustained sequence replication (Guatelli, J. C. et al., 1990, Proc. Natl. Acad. Sci. USA 87:1874-1878), transcriptional amplification system (Kwoh, D. Y. et al., 1989, Proc. Natl. Acad. Sci. USA 86:1173-1177), Q-Beta Replicase (Lizardi, P. M. et al., 1988, Bio/Technology 6:1197), and self-sustained sequence replication (Guatelli et al., (1989) Proc. Nat. Acad. Sci. 87:1874), and nucleic acid based sequence amplification (NABSA), or any other nucleic acid amplification method, followed by the detection of the amplified molecules using techniques well known to those of skill in the art. These detection schemes are especially useful for the detection of nucleic acid molecules if such molecules are present in very low numbers.

In one embodiment, any of a variety of sequencing reactions known in the art can be used to directly sequence at least a portion of a gene listed in Tables 1-5 and detect allelic variants, e.g., mutations, by comparing the sequence of the sample sequence with the corresponding reference (control) sequence. Exemplary sequencing reactions include those based on techniques developed by Maxam and Gilbert (Proc. Natl Acad Sci USA (1977) 74:560) or Sanger (Sanger et al. (1977) Proc. Nat. Acad. Sci 74:5463). It is also contemplated that any of a variety of automated sequencing procedures may be utilized when performing the subject assays (Biotechniques (1995) 19:448), including sequencing by mass spectrometry (see, for example, U.S. Pat. No. 5,547,835 and international patent application Publication Number WO 94/16101, entitled DNA Sequencing by Mass Spectrometry by H. Köster; U.S. Pat. No. 5,547,835 and international patent application Publication Number WO 94/21822 entitled “DNA Sequencing by Mass Spectrometry Via Exonuclease Degradation” by H. Köster), and U.S. Pat. No.5,605,798 and International Patent Application No. PCT[US96/03651 entitled DNA Diagnostics Based on Mass Spectrometry by H. Köster; Cohen et al. (1996) Adv Chromatogr 36:127-162; and Griffin et al. (1993) Appl Biochem Biotechnol 38:147-159). It will be evident to one skilled in the art that, for certain embodiments, the occurrence of only one, two or three of the nucleic acid bases need be determined in the sequencing reaction. For instance, A-track or the like, e.g., where only one nucleotide is detected, can be carried out.

Yet other sequencing methods are disclosed, e.g., in U.S. Pat. No. 5,580,732 and U.S. Pat. No. 5,571,676.

In some cases, the presence of a specific allele of a gene listed in Tables 1-5 in DNA from a subject can be shown by restriction enzyme analysis. For example, a specific nucleotide polymorphism can result in a nucleotide sequence comprising a restriction site which is absent from the nucleotide sequence of another allelic variant.

In a further embodiment, protection from cleavage agents (such as a nuclease, hydroxylamine or osmium tetroxide and with piperidine) can be used to detect mismatched bases in RNA/RNA DNA/DNA, or RNA/DNA heteroduplexes (Myers, et al. (1985) Science 230:1242). In general, the technique of “mismatch cleavage” starts by providing heteroduplexes formed by hybridizing a control nucleic acid, which is optionally labeled, e.g., RNA or DNA, comprising a nucleotide sequence of an allelic variant with a sample nucleic acid, e.g., RNA or DNA, obtained from a tissue sample. The double-stranded duplexes are treated with an agent which cleaves single-stranded regions of the duplex such as duplexes formed based on basepair mismatches between the control and sample strands. For instance, RNA/DNA duplexes can be treated with RNase and DNA/DNA hybrids treated with SI nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine whether the control and sample nucleic acids have an identical nucleotide sequence or in which nucleotides they are different. See, for example, Cotton et al. (1988) Proc. Natl Acad Sci USA 85:4397; Saleeba et al (1992) Methods Enzymol. 217:286-295. In a preferred embodiment, the control or sample nucleic acid is labeled for detection.

In another embodiment, an allelic variant can be identified by denaturing high-performance liquid chromatography (DHPLC) (Oefner and Underhill, (1995) Am. J. Human Gen. 57:Suppl. A266). DHPLC uses reverse-phase ion-pairing chromatography to detect the heteroduplexes that are generated during amplification of PCR fragments from individuals who are heterozygous at a particular nucleotide locus within that fragment (Oefner and Underhill (1995) Am. J. Human Gen. 57:Suppl. A266). In general, PCR products are produced using PCR primers flanking the DNA of interest. DHPLC analysis is carried out and the resulting chromatograms are analyzed to identify base pair alterations or deletions based on specific chromatographic profiles (see O'Donovan et al. (1998) Genomics 52:44-49).

In other embodiments, alterations in electrophoretic mobility is used to identify the type of allelic variant. For example, single strand conformation polymorphism (SSCP) may be used to detect differences in electrophoretic mobility between mutant and wild type nucleic acids (Orita et al. (1989) Proc Natl. Acad. Sci USA 86:2766; see also Cotton (1993) Mutat Res 285:125-144; and Hayashi (1992) Genet Anal Tech Appl 9:73-79). Single-stranded DNA fragments of sample and control nucleic acids are denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In another preferred embodiment, the subject method utilizes heteroduplex analysis to separate double stranded heteroduplex molecules on the basis of changes in electrophoretic mobility (Keen et al. (1991) Trends Genet 7:5).

In yet another embodiment, the identity of an allelic variant of a polymorphic region is obtained by analyzing the movement of a nucleic acid comprising the polymorphic region in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing agent gradient to identify differences in the mobility of control and sample DNA (Rosenbaum and Reissner (1987) Biophys Chem 265:1275).

Examples of techniques for detecting differences of at least one nucleotide between 2 nucleic acids include, but are not limited to, selective oligonucleotide hybridization, selective amplification, or selective primer extension. For example, oligonucleotide probes may be prepared in which the known polymorphic nucleotide is placed centrally (allele-specific probes) and then hybridized to target DNA under conditions which permit hybridization only if a perfect match is found (Saiki et al. (1986) Nature 324:163); Saiki et al (1989) Proc. Natl Acad. Sci USA 86:6230; and Wallace et al. (1979) Nucl. Acids Res. 6:3543). Such allele specific oligonucleotide hybridization techniques may be used for the simultaneous detection of several nucleotide changes in different polylmorphic regions of a gene listed in Tables 1-5. For example, oligonucleotides having nucleotide sequences of specific allelic variants are attached to a hybridizing membrane and this membrane is then hybridized with labeled sample nucleic acid. Analysis of the hybridization signal will then reveal the identity of the nucleotides of the sample nucleic acid.

Alternatively, allele specific amplification technology which depends on selective PCR amplification may be used in conjunction with the instant invention. Oligonucleotides used as primers for specific amplification may carry the allelic variant of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238; Newton et al. (1989) Nucl. Acids Res. 17:2503). This technique is also termed “PROBE” for Probe Oligo Base Extension. In addition it may be desirable to introduce a novel restriction site in the region of the mutation to create cleavage-based detection (Gasparini et al. (1992) Mol. Cell Probes 6:1).

In another embodiment, identification of the allelic variant is carried out using an oligonucleotide ligation assay (OLA), as described, e.g., in U.S. Pat. No. 4,998,617 and in Landegren, U. et al., (1988) Science 241:1077-1080. The OLA protocol uses two oligonucleotides which are designed to be capable of hybridizing to abutting sequences of a single strand of a target. One of the oligonucleotides is linked to a separation marker, e.g., biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson, D. A. et al. have described a nucleic acid detection assay that combines attributes of PCR and OLA (Nickerson, D. A. et al., (1990) Proc. Natl. Acad. Sci. (U.S.A.) 87:8923-8927. In this method, PCR is used to achieve the exponential amplification of target DNA, which is then detected using OLA.

Several techniques based on this OLA method have been developed and can be used to detect specific allelic variants of a polymorphic region of a gene listed in Tables 1-5. For example, U.S. Pat. No. 5593826 discloses an OLA using an oligonucleotide having 3′-amino group and a 5′-phosphorylated oligonucleotide to form a conjugate having a phosphoramidate linkage. In another variation of OLA described in Tobe et al. ((1996) Nucleic Acids Res 24: 3728), OLA combined with PCR permits typing of two alleles in a single microtiter well. By marking each of the allele-specific primers with a unique hapten, i.e. digoxigenin and fluorescein, each OLA reaction can be detected by using hapten specific antibodies that are labeled with different enzyme reporters, alkaline phosphatase or horseradish peroxidase. This system permits the detection of the two alleles using a high throughput format that leads to the production of two different colors.

The invention further provides methods for detecting single nucleotide polymorphisms in a gene listed in Tables 1-5. Because single nucleotide polymorphisms constitute sites of variation flanked by regions of invariant sequence, their analysis requires no more than the determination of the identity of the single nucleotide present at the site of variation and it is unnecessary to determine a complete gene sequence for each subject. Several methods have been developed to facilitate the analysis of such single nucleotide polymorphisms.

In one embodiment, the single base polymorphism can be detected by using a specialized exonuclease-resistant nucleotide, as disclosed, e.g., in Mundy, C. R. (U.S. Pat. No. 4,656,127). According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

In another embodiment of the invention, a solution-based method is used for determining the identity of the nucleotide of a polymorphic site (Cohen, D. et al. (French Patent 2,650,840; PCT Application No. WO91/02087). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

An alternative method, known as Genetic Bit Analysis or GBA™ is described by Goelet, P. et al. (PCT Application No. 92/15712). The method of Goelet, P. et al. uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Appln. No. WO91/02087) the method of Goelet, P. et al. is preferably a heterogeneous phase assay, in which the primer or the target molecule is immobilized to a solid phase.

Several primer-guided nucleotide incorporation procedures for assaying polymorphic sites in DNA have been described (Komher, J. S. et al., Nucl. Acids. Res. 17:7779-7784 (1989); Sokolov, B. P., Nucl. Acids Res. 18:3671 (1990); Syvanen, A. -C., et al., Genomics 8:684-692 (1990); Kuppuswamy, M. N. et al., Proc. Natl. Acad. Sci. (U.S.A.) 88:1143-1147 (1991); Prezant, T. R. et al., Hum. Mutat. 1:159-164 (1992); Ugozzoli, L. et al., GATA 9:107-112 (1992); Nyren, P. et al., Anal. Biochem. 208:171-175 (1993)). These methods differ from GBA™ in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A. -C., et al., Amer. J. Hum. Genet. 52:46-59 (1993)).

For determining the identity of the allelic variant of a polymorphic region located in the coding region of a gene listed in Tables 1-5, yet other methods than those described above can be used. For example, identification of an allelic variant which encodes a mutated protein can be performed by using an antibody specifically recognizing the mutant protein in, e.g., immunohistochemistry or immunoprecipitation. Antibodies to wild-type or mutated forms of proteins encoded by genes listed in Tables 1-5 can be prepared according to methods known in the art.

Alternatively, one can also measure an activity of a protein encoded by a gene listed in Tables 1-5, such as binding to a ligand of a protein encoded by a gene listed in Tables 1-5. Binding assays are known in the art and involve, e.g., obtaining cells from a subject, and performing binding experiments with a labeled ligand, to determine whether binding to the mutated form of the protein differs from binding to the wild-type of the protein.

Antibodies directed against reference or mutant polypeptides encoded by a gene listed in Tables 1-5 or allelic variant thereof, which are discussed above, may also be used in disease diagnostics and prognostics. Such diagnostic methods, may be used to detect abnormalities in the level of polypeptide expression, or abnormalities in the structure and/or tissue, cellular, or subcellular location of a polypeptide. Structural differences may include, for example, differences in the size, electronegativity, or antigenicity of the mutant polypeptide encoded by a gene listed in Tables 1-5 relative to the normal polypeptide encoded by a gene listed in Tables 1-5. Protein from the tissue or cell type to be analyzed may easily be detected or isolated using techniques which are well known to one of skill in the art, including but not limited to Western blot analysis. For a detailed explanation of methods for carrying out Western blot analysis, see Sambrook et al, 1989, supra, at Chapter 18. The protein detection and isolation methods employed herein may also be such as those described in Harlow and Lane, for example (Harlow, E. and Lane, D., 1988, “Antibodies: A Laboratory Manual”, Cold Spring Harbor Laboratory Press, Cold Spring Harbor, N.Y.), which is incorporated herein by reference in its entirety.

This can be accomplished, for example, by immunofluorescence techniques employing a fluorescently labeled antibody (see below) coupled with light microscopic, flow cytometric, or fluorimetric detection. The antibodies (or fragments thereof) useful in the present invention may, additionally, be employed histologically, as in immunofluorescence or immunoelectron microscopy, for in situ detection of a polypeptide encoded by a gene listed in Tables 1-5. In situ detection may be accomplished by removing a histological specimen from a subject, and applying thereto a labeled antibody of the present invention. The antibody (or fragment) is preferably applied by overlaying the labeled antibody (or fragment) onto a biological sample. Through the use of such a procedure, it is possible to determine not only the presence of the polypeptide, but also its distribution in the examined tissue. Using the present invention, one of ordinary skill will readily perceive that any of a wide variety of histological methods (such as staining procedures) can be modified in order to achieve such in situ detection.

Often a solid phase support or carrier is used as a support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads. Those skilled in the art will know many other suitable carriers for binding antibody or antigen, or will be able to ascertain the same by use of routine experimentation.

One means for labeling an antibody specific for a protein encoded by a gene listed in Tables 1-5 is via linkage to an enzyme and use in an enzyme immunoassay (EIA) (Voller, “The Enzyme Linked Immunosorbent Assay (ELISA)”, Diagnostic Horizons 2:1-7, 1978, Microbiological Associates Quarterly Publication, Walkersville, Md.; Voller, et al., J. Clin. Pathol. 31:507-520 (1978); Butler, Meth. Enzymol. 73:482-523 (1981); Maggio, (ed.) Enzyme Immunoassay, CRC Press, Boca Raton, Fla., 1980; Ishikawa, et al., (eds.) Enzyme Immunoassay, Kgaku Shoin, Tokyo, 1981). The enzyme which is bound to the antibody will react with an appropriate substrate, preferably a chromogenic substrate, in such a manner as to produce a chemical moiety which can be detected, for example, by spectrophotometric, fluorimetric or by visual means. Enzymes which can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. The detection can be accomplished by colorimetric methods which employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays. For example, by radioactively labeling the antibodies or antibody fragments, it is possible to detect fingerprint gene wild type or mutant peptides through the use of a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a gamma counter or a scintillation counter or by autoradiography.

It is also possible to label the antibody with a fluorescent compound. When the fluorescently labeled antibody is exposed to light of the proper wave length, its presence can then be detected due to fluorescence. Among the most commonly used fluorescent labeling compounds are fluorescein isothiocyanate, rhodamine, phycoerythrin, phycocyanin, allophycocyanin, o-phthaldehyde and fluorescamine.

The antibody can also be detectably labeled using fluorescence emitting metals such as ¹⁵²Eu, or others of the lanthanide series. These metals can be attached to the antibody using such metal chelating groups as diethylenetriaminepentacetic acid (DTPA) or ethylenediaminetetraacetic acid (EDTA).

The antibody also can be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

Likewise, a bioluminescent compound may be used to label the antibody of the present invention. Bioluminescence is a type of chemiluminescence found in biological systems in, which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

If a polymorphic region is located in an exon, either in a coding or non-coding portion of the gene, the identity of the allelic variant can be determined by determining the molecular structure of the mRNA, pre-mRNA, or cDNA. The molecular structure can be determined using any of the above described methods for determining the molecular structure of the genomic DNA.

The methods described herein may be performed, for example, by utilizing pre-packaged diagnostic kits, such as those described above, comprising at least one probe or primer nucleic acid described herein, which may be conveniently used, e.g., to determine whether a subject has or is at risk of developing a disease associated with a specific allelic variant.

Sample nucleic acid to be analyzed by any of the above-described diagnostic and prognostic methods can be obtained from any cell type or tissue of a subject. For example, a subject's bodily fluid (e.g. blood) can be obtained by known techniques (e.g. venipuncture). Alternatively, nucleic acid tests can be performed on dry samples (e.g. hair or skin). Fetal nucleic acid samples can be obtained from maternal blood as described in International Patent Application No. WO91/07660 to Bianchi. Alternatively, amniocytes or chorionic villi may be obtained for performing prenatal testing.

Diagnostic procedures may also be performed in situ directly upon tissue sections (fixed and/or frozen) of subject tissue obtained from biopsies or resections, such that no nucleic acid purification is necessary. Nucleic acid reagents may be used as probes and/or primers for such in situ procedures (see, for example, Nuovo, G. J., 1992, PCR in situ hybridization: protocols and applications, Raven Press, NY).

In addition to methods which focus primarily on the detection of one nucleic acid sequence, profiles may also be assessed in such detection schemes. Fingerprint profiles may be generated, for example, by utilizing a differential display procedure, Northern analysis and/or RT-PCR.

B. Pharmacogenomics

Knowledge of the identity of the allele of a polymorphic region of a gene in a subject (the genetic profile of a gene listed in Tables 1-5), alone or in conjunction with information of other genetic defects associated with the same disease (the genetic profile of the particular disease) also allows selection and customization of the therapy, e.g., a particular clinical course of therapy and/or further diagnostic evaluation for a particular disease to the subject's genetic profile. For example, subjects having specific alleles as listed in Table 5 may or may not exhibit symptoms of a particular disease or be predisposed to developing symptoms of a particular disease. Further, if those subjects are symptomatic, they may or may not respond to a certain drug, e.g., a specific therapeutic used in the treatment or prevention of abnormal lipid levels, vascular disease or disorder, e.g., CAD or MI, or a metabolic disease or disorder, such as, for example, beta blocker drugs, calcium channel blocker drugs, or nitrate drugs, cholesterol modulating, e.g., raising or lowering drugs, but may respond to another. Furthermore, they may or may not respond to other treatments, including, for example, use of medical devices for treatment of a vascular disease or disorder, a metabolic disease or disorders, or abnormal lipid levels, or surgical and/or non-surgical procedures or courses of treatment. Moreover, if a subject does or does not exhibit symptoms of a particular disease, the subject may or may not benefit from further diagnostic evaluation, including, for example, use of vascular imaging devices or procedures, for example. Furthermore, knowledge of the identity of the alleles of the polymorphic regions of a gene listed in Tables 1-5, in a subject, alone or in conjunction with information of other genetic defects associated with abnormal lipid levels or diseases or disorders associated therewith, allows predictions to be made with respect to the response by a subject to a certain therapy, e.g., HRT. For example, if a subject has alleles associated with abnormally low HDL-C, the subject's response to treatment with HRT may be a decrease in HDL-C level.

Thus, generation of a genetic profile of a gene listed in Tables 1-5, (e.g., categorization of alterations in a gene listed in Tables 1-5 which are associated with the development of a particular disease, e.g., those alleles listed in Table 5), from a population of subjects, who are symptomatic for a disease or condition that is caused by or contributed to by a defective and/or deficient gene and/or protein (a genetic population profile) and comparison of a subject's genetic profile to the population profile, permits the selection or design of drugs that are expected to be safe and efficacious for a particular subject or subject population (i.e., a group of subjects having the same genetic alteration), as well as the selection or design of a particular clinical course of therapy or further diagnostic evaluations that are expected to be safe and efficacious for a particular subject or subject population.

For example, a population profile can be performed by determining the specific gene profile, e.g., the identity of specific alleles listed in Table 5, in a subject population having a disease, which is associated with one or more specific alleles of the polymorphic regions of the gene. Optionally, the population profile can further include information relating to the response of the population to a therapeutic specific to the gene, using any of a variety of methods, including, monitoring: 1) the severity of symptoms associated with the abnormal lipid levels or vascular or metabolic diseases or disorders; 2) gene expression level; 3) mRNA level; and/or 4) protein level, and dividing or categorizing the population based on particular alleles. The genetic population profile can also, optionally, indicate those particular alleles which are present in subjects that are either responsive or non-responsive to a particular therapeutic, clinical course of therapy, or diagnostic evaluation. This information or population profile, is then useful for predicting which individuals should respond to particular drugs, particular clinical courses of therapy, or diagnostic evaluations based on their individual genetic profile of the specific gene.

In a preferred embodiment, the genetic profile is a transcriptional or expression level profile and is comprised of determining the expression level of proteins encoded by the specific gene, alone or in conjunction with the expression level of other genes known to contribute to the same disease at various stages of the disease.

Pharmacogenomic studies can also be performed using transgenic animals. For example, one can produce transgenic mice, e.g., as described herein, which contain a specific allelic variant of a gene listed in Tables 1-5. These mice can be created, e.g., by replacing their wild-type gene with an allele of the human gene listed in Tables 1-5. The response of these mice to specific particular therapeutics, clinical courses of treatment, and/or diagnostic evaluations can then be determined.

(i) Diagnostic Evaluation

In one embodiment, the polymorphisms of the present invention are used to determine the most appropriate diagnostic evaluation and to determine whether or not a subject will benefit from further diagnostic evaluation. Thus, in one embodiment, the invention provides methods for classifying a subject who has, or is at risk for developing abnormal lipid levels, e.g., abnormally low HDL-C level, or a disease or disorder associated with abnormal lipid levels, e.g., a vascular or metabolic disease or disorder, as a candidate for further diagnostic evaluation for a disease or disorder comprising the steps of determining the genetic profile of the subject, comparing the subject's genetic profile to the genetic profile of a gene listed in Tables 1-5, and classifying the subject based on the identified genetic profiles as a subject who is a candidate for further diagnostic evaluation for abnormal lipid levels, e.g., abnormally low HDL-C level, or a disease or disorder associated with abnormal lipid levels, e.g., a vascular or metabolic disease or disorder. The invention also provides methods for classifying a subject as a candidate for treatment with HRT.

In a preferred embodiment, the subject's genetic profile is determined by identifying the nucleotides present at the nucleotide positions of the SNPs set forth in Tables 1-5, wherein the presence of the specific alleles listed in Table 5, for example, indicates that a subject has, or is at increased risk for abnormal lipid levels, e.g. low HDL-C levels.

Methods of further diagnostic evaluation include use of vascular imaging devices or procedures such as, for example, angiography, cardiac ultrasound, coronary angiogram, magnetic resonance imagery, nuclear imaging, CT scan, myocardial perfusion imagery, or electrocardiogram, or may include genetic analysis, familial health history analysis, lifestyle analysis, exercise stress tests, or any combination thereof.

In another embodiment, the invention provides methods for selecting an effective imaging device as a diagnostic tool for abnormal lipid levels, e.g., abnormally low HDL-C level, or a disease or disorder associated with abnormal lipid levels, e.g., a vascular or metabolic disease or disorder, comprising the steps of determining the genetic profile of the subject; comparing the subject's genetic profile to a genetic profile of a gene listed in Tables 1-5; and selecting an effective imaging device or procedure as a diagnostic tool for abnormal lipid levels, or a vascular or metabolic disease or disorder. In a preferred embodiment, the imaging device is selected from the group consisting of angiography, cardiac ultrasound, coronary angiogram, magnetic resonance imagery, nuclear imaging, CT scan, myocardial perfusion imagery, electrocardiogram, plaque imaging, or any combination thereof.

(ii) Clinical Course of Therapy

In another aspect, the polymorphisms of the present invention are used to determine the most appropriate clinical course of therapy for a subject who has or is at risk of abnormal lipid levels, e.g., abnormally low HDL-C level, or a disease or disorder associated with abnormal lipid levels, e.g., a vascular or metabolic disease or disorder, and will aid in the determination of whether the subject will benefit from such clinical course of therapy, as determined by identification of the polymorphisms of the invention. If a subject has at any of the alleles listed in Table 5, or the complements thereof, that subject is more likely to have or to be at a higher than normal risk of developing abnormal lipid levels, e.g., low HDL-C levels, or a disease or disorder associated with abnormal lipid levels, e.g., a vascular disease or disorder or a metabolic disease or disorder.

Thus, in one aspect, the invention relates to the SNPs identified as described herein, in combination, as well as to the use of these SNPs, and others in these genes, particularly those nearby in linkage disequilibrium with these SNPs, in combination, for prediction of a particular clinical course of therapy for a subject who has, or is at risk for developing, abnormal lipid levels, e.g., abnormally low HDL-C level, or a disease or disorder associated with abnormal lipid levels, e.g., a vascular or metabolic disease or disorder. In one embodiment, the invention provides a method for determining whether a subject will benefit from a particular course of therapy by determining the presence of the polymorphisms of the invention. For example, the determination of the polymorphisms of the invention, in combination with each other, or in combination with other polymorphisms in a gene listed in Tables 1-5, or other genes, will aid in the determination of whether an individual will benefit from, for example, surgical revascularization and/or will benefit by the implantation of a stent following surgical revascularization, and will aid in the determination of the likelihood of success or failure of a particular clinical course of therapy.

In one embodiment, the invention provides methods for classifying a subject who has, or is at risk for developing, abnormal lipid levels, e.g., abnormally low HDL-C level, or a disease or disorder associated with abnormal lipid levels, e.g., a vascular or metabolic disease or disorder as a candidate for a particular clinical course of therapy for a vascular disease or disorder comprising the steps of determining the genetic profile of the subject; comparing the subject's genetic profile to a gene listed in Tables 1-5 genetic population profile; and classifying the subject based on the identified genetic profiles as a subject who is a candidate for a particular clinical course of therapy for a abnormal lipid levels or a vascular disease or disorder.

In another embodiment, the invention provides methods for selecting an effective clinical course of therapy to treat a subject who has, or is at risk for developing, abnormal lipid levels, e.g., abnormally low HDL-C level, or a disease or disorder associated with abnormal lipid levels, e.g., a vascular or metabolic disease or disorder comprising the steps of: determining the genetic profile of the subject; comparing the subject's genetic profile to the genetic profile of a gene listed in Tables 1-5; and selecting an appropriate clinical course of therapy for treatment of a subject who has, or is at risk for developing, abnormal lipid levels, e.g., abnormally low HDL-C level, or a disease or disorder associated with abnormal lipid levels, e.g., a vascular or metabolic disease or disorder.

An appropriate clinical course of therapy may include, for example, a lifestyle change, including, for example, a change in diet, exercise, or enviromnent. Other clinical courses of therapy include, but are not limited to, use of surgical procedures or medical devices. Surgical procedures for the treatment of vascular disorders, includes, for example, surgical revascularization, such as angioplasty, e.g., percutaneous transluminal coronary balloon angioplasty (PTCA), or laser angioplasty, or coronary bypass grafting (CABG). Medical devices used in the treatment or prevention of vascular diseases or disorders, include, for example, devices used in angioplasty, such as balloon angioplasty or laser angioplasty, a device used in coronary revascularization, or a stent, a defibrillator, a pacemaker, or any combination thereof. Medical devices may also be used in combination with modulators of gene expression or protein activity.

C. Monitoring Effects of Therapeutics During Clinical Trials

The present invention provides a method for monitoring the effectiveness of treatment of a subject with a therapeutic e.g., a modulator or agent of a gene listed in Tables 1-5 or a polypeptide encoded by a gene listed in Tables 1-5 (e.g., an agonist, antagonist, such as, for example, a peptidomimetic, protein, peptide, nucleic acid, ribozyme, small molecule, or other drug candidate identified, e.g., by the screening assays described herein) comprising the steps of (i) obtaining a preadministration sample from a subject prior to administration of the agent; (ii) detecting the level of expression or activity of a protein encoded by a gene listed in Tables 1-5, mRNA or gene listed in Tables 1-5 in the preadministration sample; (iii) obtaining one or more post-administration samples from the subject; (iv) detecting the level of expression or activity of the protein encoded by a gene listed in Tables 1-5, mRNA or gene listed in Tables 1-5 in the post-administration samples; (v) comparing the level of expression or activity of the protein encoded by a gene listed in Tables 1-5, mRNA, or gene listed in Tables 1-5 in the preadministration sample with those of the protein encoded by a gene listed in Tables 1-5, mRNA, or gene listed in Tables 1-5 in the post administration sample or samples; and (vi) altering the administration of the agent to the subject accordingly. For example, increased administration of the agent may be desirable to increase the expression or activity of the gene listed in Tables 1-5 to higher levels than detected, i.e., to increase the effectiveness of the agent. Alternatively, decreased administration of the agent may be desirable to decrease expression or activity of the gene to lower levels than detected, i.e., to decrease the effectiveness of the agent.

Cells of a subject may also be obtained before and after administration of a therapeutic to detect the level of expression of genes other than a gene listed in Tables 1-5, to verify that the therapeutic does not increase or decrease the expression of genes which could be deleterious. This can be done, e.g., by using the method of transcriptional profiling. Thus, mRNA from cells exposed in vivo to a therapeutic and mRNA from the same type of cells that were not exposed to the therapeutic could be reverse transcribed and hybridized to a chip containing DNA from numerous genes, to thereby compare the expression of genes in cells treated and not treated with a therapeutic. If, for example a therapeutic turns on the expression of a proto-oncogene in a subject, use of this particular therapeutic may be undesirable.

D. Methods of Treatment

The present invention provides for both prophylactic and therapeutic methods of treating a subject having or likely to develop a disorder associated with specific alleles and/or aberrant expression or activity, e.g., abnormal lipid levels, e.g., abnormally low HDL-C level, or a disease or disorder associated with abnormal lipid levels, e.g., a vascular or metabolic disease or disorder.

i) Prophylactic Methods

In one aspect, the invention provides a method for preventing a disease or disorder associated with a specific allele such as abnormal lipid levels, e.g., abnormally low HDL-C level, or a disease or disorder associated with abnormal lipid levels, e.g., a vascular or metabolic disease or disorder, and medical conditions resulting therefrom, by administering to the subject an agent which counteracts the unfavorable biological effect of the specific allele. Subjects at risk for such a disease can be identified by a diagnostic or prognostic assay, e.g., as described herein. Administration of a prophylactic agent can occur prior to the manifestation of symptoms associated with specific alleles, such that a disease or disorder is prevented or, alternatively, delayed in its progression. Depending on the identity of the allele in a subject, a compound that counteracts the effect of this allele is administered. The compound can be a compound modulating the activity of a polypeptide encoded by a gene listed in Tables 1-5, e.g., an inhibitor of a polypeptide encoded by a gene listed in Tables 1-5. The treatment can also be a specific lifestyle change, e.g., a change in diet, exercise, or an environmental alteration. In particular, the treatment can be undertaken prophylactically, before any other symptoms are present. Such a prophylactic treatment could thus prevent the development of abnormal lipid levels, e.g., abnormally low HDL-C level, or a disease or disorder associated with abnormal lipid levels, e.g., a vascular or metabolic disease or disorder. The prophylactic methods are similar to therapeutic methods of the present invention and are further discussed in the following subsections.

(ii) Therapeutic Methods

The invention further provides methods of treating a subject having a disease or disorder associated with a specific allelic variant of a polymorphic region of a gene listed in Tables 1-5, including abnormal lipid levels, e.g., abnormally low HDL-C level, or a disease or disorder associated with abnormal lipid levels, e.g., a vascular or metabolic disease or disorder.

In one embodiment, the method comprises (a) determining the identity of one or more of the allelic variants of a gene listed in Tables 1-5, or preferably, the identity of the nucleotides at the specific variant nucleotide positions, or the complements thereof, and (b) administering to the subject a compound that compensates for the effect of the specific allelic variant(s). The polymorphic region can be localized at any location of the gene, e.g., in a regulatory element (e.g., in a 5′ upstream regulatory element), in an exon, (e.g., coding region of an exon), in an intron, at an exon/intron border, or in the 3′ UTR. Thus, depending on the site of the polymorphism in a gene listed in Tables 1-5, a subject having a specific variant of the polymorphic region which is associated with a specific disease or condition, can be treated with compounds which specifically compensate for the effect of the allelic variant.

In a preferred embodiment, the identity of the nucleotides present at any of the polymorphic sites listed in Tables 1-5, or the complement thereof, is determined. For example, if a subject has any of the alleles listed in Table 5, or the complements thereof, that subject is more likely to have or to be at a higher than normal risk of developing abnormal lipid levels, e.g., low HDL-C levels, or a disease or disorder associated with abnormal lipid levels, e.g., a vascular disease or disorder or a metabolic disease or disorder.

A mutation can be a substitution, deletion, and/or addition of at least one nucleotide relative to the wild-type allele (i.e., the reference sequence). Depending on where the mutation is located in the gene, the subject can be treated to specifically compensate for the mutation. For example, if the mutation is present in the coding region of the gene and results in a more active protein, the subject can be treated, e.g., by administration to the subject of a modulator, e.g., a therapeutic or course of clinical treatment which treat, prevents, or abnormal lipid levels, e.g., abnormally low HDL-C level, or a disease or disorder associated with abnormal lipid levels, e.g., a vascular or metabolic disease or disorder. Normal protein can also be used to counteract or compensate for the endogenous mutated form of the protein encoded by a gene listed in Tables 1-5. Normal protein can be directly delivered to the subject or indirectly by gene therapy wherein some cells in the subject are transformed or transfected with an expression construct encoding wild-type protein. Nucleic acids encoding reference human proteins of the invention are set forth in SEQ ID NOs: 27-45.

Yet in another embodiment, the invention provides methods for treating a subject having a mutated gene listed in Tables 1-5, in which the mutation is located in a regulatory region of the gene. Such a regulatory region can be localized in the 5′ upstream regulatory element of the gene, in the 5′ or 3′ untranslated region of an exon, or in an intron. A mutation in a regulatory region can result in increased production of protein encoded by a gene listed in Tables 1-5, decreased production of the protein encoded by a gene listed in Tables 1-5, or production of protein encoded by a gene listed in Tables 1-5 having an aberrant tissue distribution. The effect of a mutation in a regulatory region upon the protein can be determined, e.g., by measuring the protein level or mRNA level in cells having a gene listed in Tables 1-5 having this mutation and which, normally (i.e., in the absence of the mutation) produce the protein. The effect of a mutation can also be determined in vitro. For example, if the mutation is in the 5′ upstream regulatory element, a reporter construct can be constructed which comprises the mutated 5′ upstream regulatory element linked to a reporter gene, the construct transfected into cells, and comparison of the level of expression of the reporter gene under the control of the mutated 5′ upstream regulatory element and under the control of a wild-type 5′ upstream regulatory element. Such experiments can also be carried out in mice transgenic for the mutated 5′ upstream regulatory element. If the mutation is located in an intron, the effect of the mutation can be determined, e.g., by producing transgenic animals in which the mutated gene has been introduced and in which the wild-type gene may have been knocked out. Comparison of the level of expression of a gene listed in Tables 1-5 in the mice transgenic for the mutant human gene listed in Tables 1-5 with mice transgenic for a wild-type human gene listed in Tables 1-5 will reveal whether the mutation results in increased, or decreased synthesis of the protein and/or aberrant tissue distribution of the protein. Such analysis could also be performed in cultured cells, in which the human mutant gene is introduced and, e.g., replaces the endogenous wild-type gene in the cell. Thus, depending on the effect of the mutation in a regulatory region of a gene listed in Tables 1-5, a specific treatment can be administered to a subject having such a mutation. Accordingly, if the mutation results in increased protein levels, the subject can be treated by administration of a compound which reduces protein production, e.g., by reducing gene expression or a compound which inhibits or reduces the activity of the protein encoded by a gene listed in Tables 1-5.

A correlation between drug responses and specific alleles of a gene listed in Tables 1-5 can be shown, for example, by clinical studies wherein the response to specific drugs of subjects having different allelic variants of a polymorphic region of a gene listed in Tables 1-5 is compared. Such studies can also be performed using animal models, such as mice having various alleles of a human gene listed in Tables 1-5 and in which, e.g., the endogenous gene has been inactivated such as by a knock-out mutation. Test drugs are then administered to the mice having different human alleles and the response of the different mice to a specific compound is compared. Accordingly, the invention provides assays for identifying the drug which will be best suited for treating a specific disease or condition in a subject. For example, it will be possible to select drugs which will be devoid of toxicity, or have the lowest level of toxicity possible for treating a subject having a disease or condition.

Other Uses For the Nucleic Acid Molecules of the Invention

The identification of different alleles of a gene listed in Tables 1-5 can also be useful for identifying an individual among other individuals from the same species. For example, DNA sequences can be used as a fingerprint for detection of different individuals within the same species (Thompson, J. S. and Thompson, eds., Genetics in Medicine, WB Saunders Co., Philadelphia, Pa. (1991)). This is useful, for example, in forensic studies and paternity testing, as described below.

A. Forensics

Determination of which specific allele occupies a set of one or more polymorphic sites in an individual identifies a set of polymorphic forms that distinguish the individual from others in the population. See generally National Research Council, The Evaluation of Forensic DNA Evidence (Eds. Pollard et al., National Academy Press, DC, 1996). The more polymorphic sites that are analyzed, the lower the probability that the set of polymorphic forms in one individual is the same as that in an unrelated individual. Preferably, if multiple sites are analyzed, the sites are unlinked. Thus, the polymorphisms of the invention can be used in conjunction with known polymorphisms in distal genes. Preferred polymorphisms for use in forensics are biallelic because the population frequencies of two polymorphic forms can usually be determined with greater accuracy than those of multiple polymorphic forms at multi-allelic loci.

The capacity to identify a distinguishing or unique set of polymorphic markers in an individual is useful for forensic analysis. For example, one can determine whether a blood sample from a suspect matches a blood or other tissue sample from a crime scene by determining whether the set of polymorphic forms occupying selected polymorphic sites is the same in the suspect and the sample. If the set of polymorphic markers does not match between a suspect and a sample, it can be concluded (barring experimental error) that the suspect was not the source of the sample. If the set of markers is the same in the sample as in the suspect, one can conclude that the DNA from the suspect is consistent with that found at the crime scene. If frequencies of the polymorphic forms at the loci tested have been determined (e.g., by analysis of a suitable population of individuals), one can perform a statistical analysis to determine the probability that a match of suspect and crime scene sample would occur by chance.

p(ID) is the probability that two random individuals have the same polymorphic or allelic form at a given polymorphic site. For example, in biallelic loci, four genotypes are possible: AA, AB, BA, and BB. If alleles A and B occur in a haploid genome of the organism with frequencies x and y, the probability of each genotype in a diploid organism is (see WO 95/12607):

Homozygote:p(AA)=x²

Homozygote:p(BB)=y²=(1−x)²

Single Heterozygote:p(AB)=p(BA)=xy=x(1−x)

Both Heterozygotes:p(AB+BA)=2xy=2x(1−x)

The probability of identity at one locus (i.e., the probability that two individuals, picked at random from a population will have identical polymorphic forms at a given locus) is given by the equation:

p(ID)=(x²).

These calculations can be extended for any number of polymorphic forms at a given locus. For example, the probability of identity p(ID) for a 3-allele system where the alleles have the frequencies in the population of x, y, and z, respectively, is equal to the sum of the squares of the genotype frequencies:

P(ID)=x⁴+(2xy)²+(2yz)²+(2xz)²+z⁴+y⁴.

In a locus of n alleles, the appropriate binomial expansion is used to calculate p(ID) and p(exc).

The cumulative probability of identity (cum p(ID)) for each of multiple unlinked loci is determined by multiplying the probabilities provided by each locus:

cum p(ID)=p(ID1)p(ID2)p(ID3) . . . p(IDn).

The cumulative probability of non-identity for n loci (i.e., the probability that two random individuals will be difference at 1 or more loci) is given by the equation:

cum p(nonID)=1−cum p(ID).

If several polymorphic loci are tested, the cumulative probability of non-identity for random individuals becomes very high (e.g., one billion to one). Such probabilities can be taken into account together with other evidence in determining the guilt or innocence of the suspect.

B. Paternity Testing

The object of paternity testing is usually to determine whether a male is the father of a child. In most cases, the mother of the child is known, and thus, it is possible to trace the mother's contribution to the child's genotype. Paternity testing investigates whether the part of the child's genotype not attributable to the mother is consistent to that of the putative father. Paternity testing can be performed by analyzing sets of polymorphisms in the putative father and in the child.

If the set of polymorphisms in the child attributable to the father does not match the set of polymorphisms of the putative father, it can be concluded, barring experimental error, that that putative father is not the real father. If the set of polymorphisms in the child attributable to the father does match the set of polymorphisms of the putative father, a statistical calculation can be performed to determine the probability of a coincidental match.

The probability of parentage exclusion (representing the probability that a random male will have a polymorphic form at a given polymorphic site that makes him incompatible as the father) is given by the equation (see WO 95/12607): p(exc)=xy(1−xy), where x and y are the population frequencies of alleles A and B of a biallelic polymorphic site.

(At a triallelic site p(exc)=xy(1−xy)+yz(1−yz)+xz(1−xz)+3xyz(1−xyz)), where x, y, and z and the respective populations frequencies of alleles A, B, and C).

The probability of non-exclusion is: p(non-exc)=1−p(exc).

The cumulative probability of non-exclusion (representing the values obtained when n loci are is used) is thus:

Cum p(non-exc)=p(non-exc1)p(non-exc2)p(non-exc3) . . . p(non-excn).

The cumulative probability of the exclusion for n loci (representing the probability that a random male will be excluded: cum p(exc)=1−cum p(non-exc).

If several polymorphic loci are included in the analysis, the cumulative probability of exclusion of a random male is very high. This probability can be taken into account in assessing the liability of a putative father whose polymorphic marker set matches the child's polymorphic marker set attributable to his or her father.

C. Kits

As set forth herein, the invention provides methods, e.g. diagnostic and therapeutic methods, e.g., for determining the type of allelic variant of a polymorphic region present in a gene listed in Tables 1-5, such as a human gene listed in Tables 1-5. In preferred embodiments, the methods use probes or primers comprising nucleotide sequences which are complementary to a polymorphic region of a gene listed in Tables 1-5 (SEQ ID NOs: 1-26). In a preferred embodiment, the methods use probes or primers comprising nucleotide sequences which are complementary to a polymorphic region of a gene listed in Tables 1-5. Accordingly, the invention provides kits for performing these methods. In a preferred embodiment, the kit comprises probes or primers comprising nucleotide sequences which are complementary to one or more of the alleles at of the SNPs listed in Table 5, or the complements thereof. For example, if a subject has any of the alleles listed in Table 5, or the complements thereof, that subject is more likely to have or to be at a higher than normal risk of developing abnormal lipid levels, e.g., abnormally low HDL-C level, or a disease or disorder associated with abnormal lipid levels, e.g., a vascular or metabolic disease or disorder.

In a preferred embodiment, the invention provides a kit for determining whether a subject has or is at risk of developing a disease or condition associated with a specific allelic variant of a polymorphic region of a gene listed in Tables 1-5. In an even more preferred embodiment, the disease or disorder is characterized by an abnormal activity of a polypeptide encoded by a gene listed in Tables 1-5. In an even more preferred embodiment, the invention provides a kit for determining whether a subject has or is or is not at risk of developing abnormal lipid levels, e.g., abnormally low HDL-C level, or a disease or disorder associated with abnormal lipid levels, e.g., a vascular or metabolic disease or disorder.

A preferred kit provides reagents for determining whether a subject is likely to develop abnormal lipid levels, e.g., abnormally low HDL-C level, or a disease or disorder associated with abnormal lipid levels, e.g., a vascular or metabolic disease or disorder.

Preferred kits comprise at least one probe or primer which is capable of specifically hybridizing under stringent conditions to a sequence or polymorphic region of a gene listed in Tables 1-5 and instructions for use. The kits preferably comprise at least one of the above described nucleic acids. Preferred kits for amplifying at least a portion of a gene listed in Tables 1-5 comprise at least two primers, at least one of which is capable of hybridizing to an allelic variant sequence.

The kits of the invention can also comprise one or more control nucleic acids or reference nucleic acids, such as nucleic acids comprising an intronic sequence. For example, a kit can comprise primers for amplifying a polymorphic region of a gene listed in Tables 1-5 and a control DNA corresponding to such an amplified DNA and having the nucleotide sequence of a specific allelic variant. Thus, direct comparison can be performed between the DNA amplified from a subject and the DNA having the nucleotide sequence of a specific allelic variant. In one embodiment, the control nucleic acid comprises at least a portion of a gene listed in Tables 1-5 of an individual who does not have abnormal lipid levels, e.g., abnormally low HDL-C level, or a disease or disorder associated with abnormal lipid levels, e.g., a vascular or metabolic disease or disorder, or a disease or disorder associated with an aberrant activity of a polypeptide encoded by a gene listed in Tables 1-5.

Yet other kits of the invention comprise at least one reagent necessary to perform the assay. For example, the kit can comprise an enzyme. Alternatively the kit can comprise a buffer or any other necessary reagent.

D. Electronic Apparatus Readable Media and Arrays

Electronic apparatus readable media comprising polymorphisms of the present invention is also provided. As used herein, “electronic apparatus readable media” and “computer readable media,” which are used interchangeably herein, refer to any suitable medium for storing, holding or containing data or information that can be read and accessed directly by an electronic apparatus. Such media can include, but are not limited to: magnetic storage media, such as floppy discs, hard disc storage medium, and magnetic tape; optical storage media such as compact disc; electronic storage media such as RAM, ROM, EPROM, EEPROM and the like; general hard disks and hybrids of these categories such as magnetic/optical storage media. The medium is adapted or configured for having recorded thereon a marker of the present invention.

As used herein, the term “electronic apparatus” is intended to include any suitable computing or processing apparatus or other device configured or adapted for storing data or information. Examples of electronic apparatus suitable for use with the present invention include stand-alone computing apparatus; networks, including a local area network (LAN), a wide area network (WAN) Internet, Intranet, and Extranet; electronic appliances such as a personal digital assistants (PDAs), cellular phone, pager and the like; and local and distributed processing systems.

As used herein, “recorded” refers to a process for storing or encoding information on the electronic apparatus readable medium. Those skilled in the art can readily adopt any of the presently known methods for recording information on known media to generate manufactures comprising the polymorphisms of the present invention.

A variety of software programs and formats can be used to store the polymorphisms information of the present invention on the electronic apparatus readable medium. For example, the polymorphic sequence can be represented in a word processing text file, formatted in commercially-available software such as WordPerfect and MicroSoft Word, or represented in the form of an ASCII file, stored in a database application, such as DB2, Sybase, Oracle, or the like, as well as in other forms. Any number of data processor structuring formats (e.g., text file or database) may be employed in order to obtain or create a medium having recorded thereon the markers of the present invention.

By providing the polymorphisms of the invention in readable form, in combination, one can routinely access the polymorphism information for a variety of purposes. For example, one skilled in the art can use the sequences of the polymorphisms of the present invention in readable form to compare a target sequence or target structural motif with the sequence information stored within the data storage means. Search means are used to identify fragments or regions of the sequences of the invention which match a particular target sequence or target motif.

The present invention therefore provides a medium for holding instructions for performing a method for determining whether a subject has abnormal lipid levels, e.g., abnormally low HDL-C level, or a disease or disorder associated with abnormal lipid levels, e.g., a vascular or metabolic disease or disorder or a pre-disposition to abnormal lipid levels, e.g., abnormally low HDL-C level, or a disease or disorder associated with abnormal lipid levels, e.g., a vascular or metabolic disease or disorder, wherein the method comprises the steps of determining the presence or absence of a polymorphism and based on the presence or absence of the polymorphism, determining whether the subject has abnormal lipid levels or a pre-disposition to abnormal lipid levels and/or recommending a particular clinical course of therapy or diagnostic evaluation for the abnormal lipid levels or pre-abnormal lipid condition.

The present invention further provides in an electronic system and/or in a network, a method for determining whether a subject has abnormal lipid levels or a pre-disposition to abnormal lipid levels associated with a polymorphism as described herein wherein the method comprises the steps of determining the presence or absence of the polymorphism, and based on the presence or absence of the polymorphism, determining whether the subject has abnormal lipid levels or a pre-disposition to abnormal lipid levels, and/or recommending a particular treatment for the abnormal lipid levels or pre-abnormal lipid condition. The method may further comprise the step of receiving phenotypic information associated with the subject and/or acquiring from a network phenotypic information associated with the subject.

The present invention also provides in a network, a method for determining whether a subject has abnormal lipid levels, e.g., abnormally low HDL-C level, or a disease or disorder associated with abnormal lipid levels, e.g., a vascular or metabolic disease or disorder or a pre-disposition to abnormal lipid levels or a vascular or metabolic disease or disorder associated with a polymorphism, said method comprising the steps of receiving information associated with the polymorphism, receiving phenotypic information associated with the subject, acquiring information from the network corresponding to the polymorphism and/or abnormal lipid levels, and based on one or more of the phenotypic information, the polymorphism, and the acquired information, determining whether the subject has abnormal lipid levels or a pre-disposition to abnormal lipid levels or a vascular or metabolic disease or disorder. The method may further comprise the step of recommending a particular treatment for the abnormal lipid levels or pre-abnormal lipid condition.

The present invention also provides a method for determining whether a subject has a pre-disposition to abnormal lipid levels, a vascular disease or disorder, or a metabolic disease or disorder, said method comprising the steps of receiving information associated with the polymorphism, receiving phenotypic information associated with the subject, acquiring information from the network corresponding to the polymorphism and/or abnormal lipid levels, and based on one or more of the phenotypic information, the polymorphism, and the acquired information, determining whether the subject has abnormal lipid levels or a pre-disposition to abnormal lipid levels, a vascular disease or disorder, or a metabolic disease or disorder. The method may further comprise the step of recommending a particular treatment for the abnormal lipid levels or pre-abnormal lipid condition.

E. Personalized Health Assessment

Methods and systems of assessing personal health and risk for disease, e.g., abnormal lipid levels, e.g., abnormally low HDL-C level, or a disease or disorder associated with abnormal lipid levels, e.g., a vascular or metabolic disease or disorder, in a subject, using the polymorphisms and association of the instant invention are also provided. The methods provide personalized health care knowledge to individuals as well as to their health care providers, as well as to health care companies. It will be appreciated that the term “health care provider” is not limited to a physician but can be any source of health care. The methods and systems provide personalized information including a personal health assessment report that can include a personalized molecular profile, e.g., a genetic profile, a health profile, or both. As used herein, the term “health assessment” includes the assessment of health by any source of health care, including, but not limited to, a physician or a health care company. Overall, the methods and systems as described herein provide personalized information for individuals and patient management tools for healthcare providers and/or subjects using a variety of communications networks such as, for example, the Internet. U.S. patent application Ser. No. 60/266,082, filed Feb. 1, 2001, entitled “Methods and Systems for Personalized Health Assessment,” further describes personalized health assessment methods, systems, and apparatus, and is expressly incorporated herein by reference.

In one aspect, the invention provides an Internet-based method for assessing a subject's risk for abnormal lipid levels, e.g., abnormally low HDL-C level, or a disease or disorder associated with abnormal lipid levels, e.g., a vascular or metabolic disease or disorder. In one embodiment, the method comprises obtaining a biological sample from a subject, analyzing the biological sample to determine the presence or absence of a polymorphic region of a gene listed in Tables 1-5, and providing results of the analysis to the subject via the Internet, wherein the presence of a polymorphic region of a gene listed in Tables 1-5 indicates an increased or decreased risk for abnormal lipid levels. In another embodiment, the method comprises analyzing data from a biological sample from a subject relating to the presence or absence of a polymorphic region of a gene listed in Tables 1-5 and providing results of the analysis to the subject via the Internet, wherein the presence of a polymorphic region of a gene listed in Tables 1-5 indicates an increased or decreased risk for having or developing an abnormal lipid level.

It will be appreciated that the phrase “wherein the presence of a polymorphic region of a gene listed in Tables 1-5 indicates an increased risk for abnormal lipid levels” includes an increased or higher than normal risk of developing an abnormal lipid levels, e.g., abnormally low HDL-C level, or a disease or disorder associated with abnormal lipid levels, e.g., a vascular or metabolic disease or disorder indicated by a subject having any of the alleles listed in Tables 1-5, preferably one of the specific alleles listed in Table 5, or the complements thereof.

The terms “Internet” and/or “communications network” as used herein refer to any suitable communication link, which permits electronic communications. It should be understood that these terms are not limited to “the Internet” or any other particular system or type of communication link. That is, the terms “Internet” and/or “communications network” refer to any suitable communication system, including extra-computer system and intra-computer system communications. Examples of such communication systems include internal busses, local area networks, wide area networks, point-to-point shared and dedicated communications, infra-red links, microwave links, telephone links, CATV links, satellite and radio links, and fiber-optic links. The terms “Internet” and/or “communications network” can also refer to any suitable communications system for sending messages between remote locations, directly or via a third party communication provider such as AT&T. In this instance, messages can be communicated via telephone or facsimile or computer synthesized voice telephone messages with or without voice or tone recognition, or any other suitable communications technique.

In another aspect, the methods of the invention also provide methods of assessing a subject's risk for abnormal lipid levels. In one embodiment, the method comprises obtaining information from the subject regarding the polymorphic region of a gene listed in Tables 1-5, through e.g., obtaining a biological sample from the individual, analyzing the sample to obtain the subject's genetic profile, representing the genetic profile information as digital genetic profile data, electronically processing the digital genetic profile data to generate a risk assessment report for abnormal lipid levels, e.g., abnormally low HDL-C level, or a disease or disorder associated with abnormal lipid levels, e.g., a vascular or metabolic disease or disorder, and displaying the risk assessment report on an output device, where the presence of a polymorphic region of a gene listed in Table 5 indicates an increased risk for abnormal lipid levels, e.g., abnormally low HDL-C level, or a disease or disorder associated with abnormal lipid levels, e.g., a vascular or metabolic disease or disorder. In another embodiment, the method comprises analyzing a subject's genetic profile, representing the genetic profile information as digital genetic profile data, electronically processing the digital genetic profile data to generate a risk assessment report for vascular disease, and displaying the risk assessment report on an output device, where the presence of a polymorphic region of a gene listed in Table 5 indicates an increased risk for abnormal lipid levels, e.g., abnormally low HDL-C level, or a disease or disorder associated with abnormal lipid levels, e.g., a vascular or metabolic disease or disorder. Additional health information may be provided and can be utilized to generate the risk assessment report. Such information includes, but is not limited to, information regarding one or more of age, sex, ethnic origin, diet, sibling health, parental health, clinical symptoms, personal health history, blood test data, weight, and alcohol use, drug use, nicotine use, and blood pressure.

The digital genetic profile data may be transmitted via a communications network, e.g., the Internet, to a medical information system for processing.

In yet another aspect the invention provides a medical information system for assessing a subject's risk for abnormal lipid levels, e.g., abnormally low HDL-C level, or a disease or disorder associated with abnormal lipid levels, e.g., a vascular or metabolic disease or disorder comprising a means for obtaining information from the subject regarding the polymorphic region of a gene listed in Tables 1-5, through e.g., obtaining a biological sample from the individual to obtain a genetic profile, a means for representing the genetic profile as digital molecular data, a means for electronically processing the digital genetic profile to generate a risk assessment report for abnormal lipid levels, e.g., abnormally low HDL-C level, or a disease or disorder associated with abnormal lipid levels, e.g., a vascular or metabolic disease or disorder, and a means for displaying the risk assessment report on an output device, where the presence of a polymorphic region of a gene listed in Table 5 indicates an increased risk for having or developing an abnormal lipid level.

In another aspect, the invention provides a computerized method of providing advice, e.g., actionable advice or medical advice, to a subject comprising obtaining information from the subject regarding the polymorphic region of a gene listed in Tables 1-5, through e.g., obtaining a biological sample from the subject, analyzing the subject's biological sample to determine the subject's genetic profile, and, based on the subject's genetic profile, determining the subject's risk for abnormal lipid levels, e.g., abnormally low HDL-C level, or a disease or disorder associated with abnormal lipid levels, e.g., a vascular or metabolic disease or disorder. Advice, e.g., actionable advice, may be then provided electronically to the subject, based on the subject's risk for abnormal lipid levels, e.g., abnormally low HDL-C level, or a disease or disorder associated with abnormal lipid levels, e.g., a vascular or metabolic disease or disorder. The advice, e.g., actionable advice, may comprise, for example, recommending one or more of the group consisting of: further diagnostic evaluation, use of medical or surgical devices, administration of medication, e.g. lipid modulating medication, or lifestyle change, e.g., diet or exercise change. Additional health information may also be obtained from the subject and may also be used to provide the advice.

In another aspect, the invention includes a method for self-assessing risk for a vascular or metabolic disease or disorder. The method comprises providing information from the subject regarding the polymorphic region of a gene listed in Tables 1-5, through e.g., providing a biological sample for genetic analysis, and accessing an electronic output device displaying results of the genetic analysis, thereby self-assessing risk for abnormal lipid levels, e.g., abnormally low HDL-C level, or a disease or disorder associated with abnormal lipid levels, e.g., a vascular or metabolic disease or disorder, where the presence of a polymorphic region of a gene listed in Table 5 indicates an increased risk for abnormal lipid levels, e.g., abnormally low HDL-C level, or a disease or disorder associated with abnormal lipid levels, e.g., a vascular or metabolic disease or disorder.

In another aspect, the invention provides a method of self-assessing risk for a vascular or metabolic disease or disorder comprising providing information from the subject regarding the polymorphic region of a gene listed in Tables 1-5, through e.g., providing a biological sample, accessing digital genetic profile data obtained from the biological sample, the digital genetic profile data being displayed via an output device, where the presence of a polymorphic region of a gene listed in Table 5 indicates an increased risk for abnormal lipid levels, e.g., abnormally low HDL-C level, or a disease or disorder associated with abnormal lipid levels, e.g., a vascular or metabolic disease or disorder.

An output device may be, for example, a CRT, printer, or website. An electronic output device may be accessed via the Internet.

The biological sample may be obtained from the individual at a laboratory company. In one embodiment, the laboratory company processes the biological sample to obtain genetic profile data, represents at least some of the genetic profile data as digital genetic profile data, and transmits the digital genetic profile data via a communications network to a medical information system for processing. The biological sample may also be obtained from the subject at a draw station. A draw station processes the biological sample to obtain genetic profile data and transfers the data to a laboratory company. The laboratory company then represents at least some of the genetic profile data as digital genetic profile data, and transmits the digital genetic profile data via a communications network to a medical information system for processing.

In another aspect, the invention provides a method for a health care provider to generate a personal health assessment report for an individual. The method comprises counseling the individual to provide a biological sample and authorizing a draw station to take a biological sample from the individual and transmit molecular information from the sample to a laboratory company, where the molecular information comprises the presence or absence of a polymorphic region of a gene listed in Tables 1-5. The health care provider then requests the laboratory company to provide digital molecular data corresponding to the molecular information to a medical information system to electronically process the digital molecular data and digital health data obtained from the individual to generate a health assessment report, receives the health assessment report from the medical information system, and provides the health assessment report to the individual.

In still another aspect, the invention provides a method of assessing the health of an individual. The method comprises obtaining health information from the individual using an input device (e.g., a keyboard, touch screen, hand-held device, telephone, wireless input device, or interactive page on a website), representing at least some of the health information as digital health data, obtaining a biological sample from the individual, and processing the biological sample to obtain molecular information, where the molecular information comprises the presence or absence of a polymorphic region of a gene listed in Tables 1-5. At least some of the molecular information and health data is then presented as digital molecular data and electronically processed to generate a health assessment report. The health assessment report is then displayed on an output device. The health assessment report can comprise a digital health profile of the individual. The molecular data can comprise protein sequence data, and the molecular profile can comprise a proteomic profile. The molecular data can also comprise information regarding one or more of the absence, presence, or level, of one or more specific proteins, polypeptides, chemicals, cells, organisms, or compounds in the individual's biological sample. The molecular data may also comprise, e.g., nucleic acid sequence data, and the molecular profile may comprise, e.g., a genetic profile.

In yet another embodiment, the method of assessing the health of an individual further comprises obtaining a second biological sample or a second health information at a time after obtaining the initial biological sample or initial health information, processing the second biological sample to obtain second molecular information, processing the second health information, representing at least some of the second molecular information as digital second molecular data and second health information as digital health information, and processing the molecular data and second molecular data and health information and second health information to generate a health assessment report. In one embodiment, the health assessment report provides information about the individual's predisposition for abnormal lipid levels, e.g., abnormally low HDL-C level, or a disease or disorder associated with abnormal lipid levels, e.g., a vascular or metabolic disease or disorder, and options for risk reduction.

Options for risk reduction comprise, for example, one or more of diet, exercise, one or more vitamins, one or more drugs, cessation of nicotine use, and cessation of alcohol use. In one embodiment, the health assessment report provides information about treatment options for a particular disorder. Treatment options comprise, for example, one or more of diet, one or more drugs, physical therapy, and surgery. In one embodiment, the health assessment report provides information about the efficacy of a particular treatment regimen and options for therapy adjustment.

In another embodiment, electronically processing the digital molecular data and digital health data to generate a health assessment report comprises using the digital molecular data and/or digital health data as inputs for an algorithm or a rule-based system that determines whether the individual is at risk for a specific disorder, e.g., abnormal lipid levels, e.g., abnormally low HDL-C level, or a disease or disorder associated with abnormal lipid levels, e.g., a vascular or metabolic disease or disorder. Electronically processing the digital molecular data and digital health data may also comprise using the digital molecular data and digital health data as inputs for an algorithm or a rule-based system based on one or more databases comprising stored digital molecular data and/or digital health data relating to one or more disorders, e.g., abnormal lipid levels, e.g., abnormally low HDL-C level, or a disease or disorder associated with abnormal lipid levels, e.g., a vascular or metabolic disease or disorder.

In another embodiment, processing the digital molecular data and digital health data comprises using the digital molecular data and digital health data as inputs for an algorithm or a rule-based system based on one or more databases comprising: (i) stored digital molecular data and/or digital health data from a plurality of healthy individuals, and (ii) stored digital molecular data and/or digital health data from one or more pluralities of unhealthy individuals, each plurality of individuals having a specific disorder. At least one of the databases can be a public database. In one embodiment, the digital health data and digital molecular data are transmitted via, e.g., a communications network, e.g., the Internet, to a medical information system for processing.

A database of stored molecular data and health data, e.g., stored digital molecular data and/or digital health data, from a plurality of individuals, is further provided. A database of stored digital molecular data and/or digital health data from a plurality of healthy individuals, and stored digital molecular data and/or digital health data from one or more pluralities of unhealthy individuals, each plurality of individuals having a specific disorder, e.g., abnormal lipid levels, e.g., abnormally low HDL-C level, or a disease or disorder associated with abnormal lipid levels, e.g., a vascular or metabolic disease or disorder, is also provided.

The new methods and systems of the invention provide healthcare providers with access to ever-growing relational databases that include both molecular data and health data that is linked to specific disorders, e.g., abnormal lipid levels, e.g., abnormally low HDL-C level, or a disease or disorder associated with abnormal lipid levels, e.g., a vascular or metabolic disease or disorder. In addition public medical knowledge is screened and abstracted to provide concise, accurate information that is added to the database on an ongoing basis. In addition, new relationships between particular SNPs, e.g., SNPs associated with abnormal lipid levels, e.g., abnormally low HDL-C level, or a disease or disorder associated with abnormal lipid levels, e.g., a vascular or metabolic disease or disorder, or genetic mutations and specific discords are added as they are discovered.

The present invention is further illustrated by the following examples which should not be construed as limiting in any way. The contents of all cited references (including, without limitation, literature references, issued patents, published patent applications and database records including Genbank™ records) as cited throughout this application are hereby expressly incorporated by reference. The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No: 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

### EXAMPLES

### Example 1

Identification of Polymorphic Regions of Genes Associated with Abnormal Lipid Levels

Population

All cases were drawn from the GeneQuest study, a collection of families with premature coronary artery disease. Subjects in the GeneQuest study all had premature coronary artery disease identified at one of 15 participating medical centers, fulfilling the criteria of either myocardial infarction, surgical or percutaneous revascularization, or a significant coronary artery lesion diagnosed before age 45 in males or age 50 in females and having a living sibling who met the same criteria. For this study, one individual per family was selected for genotyping. The final sample was comprised of 352 Caucasian individuals with a personal and family history of premature CAD.

Methods

Variant Discovery

Most of the allelic variants of the present invention were identified through denaturing high performance liquid chromatography (DHPLC) analysis, variant detector arrays (Affymetrix™), the polymerase chain reaction (PCR), and/or single stranded conformation polymorphism (SSCP) analysis using PCR primers complementary to intronic sequences surrounding each of the exons, 3′ UTR, and 5′ upstream regulatory element sequences of the genes. The polymorphic regions of the present invention have been identified in the genes by analyzing the DNA of cell lines derived from an ethnically diverse population by methods described in Cargill, et al. (1999 Nature Genetics 22:231-238).

Genotyping

Oligonucleotides primers and probes were designed using Primer Express v1.5 (Applied Biosystems, Inc.). Two types of TaqMan probes were used: “TAMRA” assays (FAM and TET reporter dyes; TAMRA quencher dye); and “MGB” assays (FAM and VIC reporter dyes; dark quencher; minor groove binding group on 3′ end).

PCR reactions contained 900 nM primers, 200 nM TET probe, 100 nM (TAMRA assays) or 200 nM (MGB assays) FAM probe, 8 ng genomic DNA and 3 uL TaqMan Universal PCR Master Mix (Applied Biosystems, Inc.) in a 5 uL reaction volume Thermocycling conditions were as follows: 1 cycle of 95° C. for10 minutes, 40 cycles of 92° C. for 15 seconds and 62° C. for 1 minute, 1 cycle of 20° C. for 1 minute. Following thermocycling, endpoint fluorescence values for allele specific probes were quantitated using a SpectroMax Gemini XS spectrophotometer (Molecular Devices, Inc.). Genotypes were assigned based on a scatter plot of normalized fluorescence values.

Statistical Analysis

The association of HDL-C with each SNP was assessed in a general linear model, implemented in the PROC GLM procedure in SAS. Each SNP was entered into the model using a class statement to define it as an unordered categorical variable. Significance was determined for the SNP, taking into account all three genotypes, with the F statistic. In those cases where the homozygous variant genotype was rare (<5 observations), heterozygous and homozygous variant genotypes were pooled. Mean and standard deviations of HDL-C are reported for each genotype.

All SNPs were evaluated alone, and in a model testing for interaction between the SNP and sex. For those SNPs where significant (p<0.05) interaction was found, mean values of HDL-C by genotype were presented separately for males and females. Significance of the association of each of these SNPs with HDL-C was assessed with the F statistic.

Multivariate analysis was carried out with a backwards stepwise procedure, beginning with all SNPs found to be significantly associated with HDL-C in univariate analyses.

Linkage disequilibrium was assessed with the normalized disequilibrium parameter, D′, using the EM algorithm.

Results

Significant associations between HDL-C and variants in nine genes (Table 2) were identified. Associations between HDL-C and variants in ten additional genes were found when males and females were analyzed separately (Table 3). These SNPs, identified through significant SNP by sex interaction, usually conferred the opposite effect in males and females. However, the effect in females was typically stronger, resulting in significant associations despite the smaller sample size.

For some genes multiple SNPs were typed. In some cases, SNPs in a gene were highly correlated, or in linkage disequilibrium, and yet not all of these SNPs showed significant (p<0.05) associations with HDL-C. Table 4 lists additional SNPs which are in linkage disequilibrium with the associated SNPs even though they themselves did not reach statistical significance. Table 5 provides a summary of the SNPs associated with abnormal HDL-C level, e.g., low HDL-C levels.

For two genes where multiple SNPs were typed, more than one SNP showed a statistically significant association with HDL-C. In the LIPC gene, both the LIPC_{—}1 and LIPC_{—}5 SNPs were associated with HDL-C. These two SNPs are not in linkage disequilibrium (D′=0.10, p=0.37). Therefore, they represent independent risk factors. Similarly, in the LRP1 gene, two SNPs were significantly associated with HDL-C, LRP 1_{—}1 and LRP1_{—}3. These SNPs are not in linkage disequilibrium either (D′=-0.13, p=0.49) and therefore represent independent associations.

Results from a multivariate analysis (Table 6 and Table 7) revealed that different genes may influence HDL-C levels in males and females. In females, five genes were independently associated with HDL-C including COL5A2, F2, CD14, VWF, and ITGB3. The combination of these five genes account for approximately 65% of the variability in HDL-C. In males, a different combination of three genes was identified. COL5A2, CD14 and FABP3 were independently associated with HDL-C and together account for approximately 21% of the variation in HDL-C in males.

TABLE 6
Final model for genes associated with HDL-C in males.

| | Parameter | Standard | | |
|---|---|---|---|---|
| Variable | Estimate | Error | F Value | Pr > F |
| Intercept | 44.46214 | 1.98821 | 500.10 | <.0001 |
| COL5A2_1_AG | 2.10359 | 2.12880 | 0.98 | 0.3253 |
| COL5A2_1_AA | 25.90332 | 7.42166 | 12.18 | 0.0007 |
| CD14_1_CT | −6.42302 | 2.31371 | 7.71 | 0.0065 |
| CD14_1_CC | −5.30791 | 2.74417 | 3.74 | 0.0557 |
| FABP3_1_TC | −10.70610 | 3.63809 | 8.66 | 0.0040 |

Male subjects having or at risk for developing the lowest levels of HDL-C are those with the following combination of genotypes: COL5A2_{—}1 GG, CD14_{—}1 CT or CC and FABP3_{—}1 CT. This combination is predicted to result in a mean HDL-C level below approximately 29 mg/dl. Based on individual genotype frequencies, this combination is estimated to have a frequency of approximately 4% in a general U.S. Caucasian population.

TABLE 7
Final model for genes associated with HDL-C in females.

| | Parameter | Standard | | |
|---|---|---|---|---|
| Variable | Estimate | Error | F Value | Pr > F |
| Intercept | 52.56006 | 3.13836 | 280.48 | <.0001 |
| COL5A2_1_AG | 9.41856 | 3.17158 | 8.82 | 0.0054 |
| COL5A2_1_AA | 10.41297 | 6.42627 | 2.63 | 0.1141 |
| CD14_1_CT | −11.89890 | 3.30875 | 12.93 | 0.0010 |
| CD14_1_CC | −4.86941 | 3.64396 | 1.79 | 0.1901 |
| VWF_2_GA | −12.28175 | 3.53269 | 12.09 | 0.0014 |
| F2_1_CT | 1.62957 | 3.35462 | 0.24 | 0.6302 |
| F2_1_TT | −25.10920 | 8.86448 | 8.02 | 0.0076 |
| ITGB3_4_TC | −11.56171 | 2.83489 | 16.63 | 0.0002 |
| ITGB3_4_TT | 20.82224 | 5.48327 | 14.42 | 0.0006 |

The frequencies of the genotypes conferring the lowest levels of HDL-C for each of these SNPs was remarkably high. One exception is for the F2_{—}1 SNP. For the F2 SNP, the homozygous variant genotype (TT), which has the effect of lowering HDL-C by 25 mg/dl, has a frequency of only 1%. For the four remaining SNPs, female subjects having or at risk for developing the lowest levels of HDL-C are those with the following combination of genotypes: COL5A2_{—}1 GG, CD14_{—}1 CT, VWF_{—}2 GA and ITGB3 4 TC. This combination is predicted to result in a mean HDL-C level of approximately 16.8 mg/dl. Based on individual genotype frequencies, this combination is estimated to have a frequency of approximately 3% in a general U.S. Caucasian population.

Equivalents

Those skilled in the art will recognize, or be able to ascertain using no more than routine experimentation, many equivalents to the specific embodiments of the invention described herein. Such equivalents are intended to be encompassed by the following claims.

What is claimed is:

1. A method for determining whether a subject has, or is at risk of developing, an abnormally low HDL-C level, comprising determining whether the subject has an allelic variant of a polymorphic region listed in Table 5, to thereby determine whether the subject has, or is at risk for developing, an abnormally low HDL-C level.

2. The method of claim 1, wherein said allelic variant is APOA_{—}1 CC, CD14_{—}1 CT, COL5A2_{—}1 GG, EDNRB_{—}1 AG or AA, FABP3_{—}1 CT, GBE1_{—}1 AG or GG, LIPC_{—}5 AA, MTHFR_{—}1 CC, VWF_{—}2 GG, or the complements thereof.

3. A method for determining whether a male subject has, or is at risk of developing, an abnormally low HDL-C level, comprising determining whether the male subject has an allelic variant of a polymorphic region listed in Table 5 which is associated with abnormally low HDL-C levels in males, to thereby determine whether the male subject has, or is at risk for developing an abnormally low HDL-C level.

4. The method of claim 3, wherein said allelic variant is LRP1_{—}3 CC or CT, PAI2_{—}4 GG, or PPARG_{—}1 CG, or the complements thereof

5. The method of claim 3, wherein said allelic variants are COL5A2_{—}1 GG, CD14_{—}1 CT or CC, and FABP3_{—}1 CT, in combination, or the complements thereof.

6. A method for determining whether a female subject has, or is at risk of developing, an abnormally low HDL-C level, comprising determining whether the female subject has an allelic variant of a polymorphic region listed in Table 5 which is associated with abnormally low HDL-C levels in females, to thereby determine whether the female subject has, or is at risk for developing an abnormally low HDL-C level.

7. The method of claim 6, wherein said allelic variant is AT3_{—}1 AG or AA, F2_{—}1 TT, ITGB3_{—}4 TC, LIPC_{—}1 AG or AA, LRP1_{—}1 GT or TT, PPARG_{—}1 CC, PRCP_{—}1 CC, THBS4_{—}1 GG or GC, or the complements thereof.

8. The method of claim 6, wherein said allelic variants are COL5A2_{—}1 GG, CD14_{—}1 CT, VWF_{—}2 GA, and ITGB3_{—}4 TC, in combination, or the complements thereof.

9. The method of claim 1, wherein determining the identity of the allelic variant of a polymorphic region comprises contacting a nucleic acid of the subject with at least one probe or primer which is capable of hybridizing to a gene listed in Table 5.

10. The method of claim 3, wherein determining the identity of the allelic variant of a polymorphic region comprises contacting a nucleic acid of the subject with at least one probe or primer which is capable of hybridizing to a gene listed in Table 5.

11. The method of claim 6, wherein determining the identity of the allelic variant of a polymorphic region comprises contacting a nucleic acid of the subject with at least one probe or primer which is capable of hybridizing to a gene listed in Table 5.

12. The method of claims 9, 10, or 11, wherein the probe or primer is capable of specifically hybridizing to an allelic variant of the polymorphic region.

13. The method of claims 9, 10, or 11, wherein the probe or primer has a nucleotide sequence from about 15 to about 30 nucleotides.

14. The method of claims 9, 10, or 11, wherein the probe or primer is a single stranded nucleic acid.

15. The method of claims 9, 10, or 11, wherein the probe or primer is labeled.

16. The method of claims 1, 3, or 6, wherein determining the identity of the allelic variant of a polymorphic region is carried out by allele specific hybridization.

17. The method of claims 1, 3, or 6, wherein determining the identity of the allelic variant of a polymorphic region is carried out by primer specific extension.

18. The method of claims 1, 3, or 6, wherein determining the identity of the allelic variant of a polymorphic region is carried out by an oligonucleotide ligation assay.

19. The method of claims 1, 3, or 6, wherein determining the identity of the allelic variant of a polymorphic region is carried out by single-stranded conformation polymorphism.
